(19) United States
(12) Patent Application Publication
(10) Pub. No.: US 2024/0054640 A1
(43) Pub. Date: Feb. 15, 2024

(54) System, Method, and Computer Program Product for Classification of Diseases Based on Expansion Microscopic Images

(71) Applicant: Carnegie Mellon University, Pittsburgh, PA (US)

(72) Inventors: Yongxin Zhao, Sewickley, PA (US); Christopher Byungjun Ahn, Loveland, OH (US)

(21) Appl. No.: 18/267,134

(22) PCT Filed: Dec. 15, 2021
(86) PCT No.: PCT/US21/63543

§ 371 Date: Jun. 14, 2023

Related U.S. Application Data
(60) Provisional application No. 63/125,434, filed on Dec. 15, 2020.

(51) Int. Cl. G06T 7/00; G06T 7/194; G06T 7/11; G06V 10/774; G06V 20/69; G06T 7/73; G06V 20/70; G06V 10/776; G06V 10/82; G06T 3/40; G16H 20/40

(52) U.S. Cl. CPC G06T 7/0012; G06T 7/194; G06T 7/11; G06V 10/774; G06V 20/698; G06T 7/73; G06V 20/70; G06V 10/776; G06V 10/82; G06T 3/4053; G06V 20/695; G16H 20/40; G06T 2207/20021; G06T 2207/20081; G06T 2207/20084; G06T 2200/24; G06T 2207/10056; G06T 2207/30024; G06V 2201/03

(57) ABSTRACT

Provided is a method for classification of diseases including receiving image data associated with an image at a first resolution. The image may be processed, for example by removing a background from the image, deconstructing the image into separate layers, and segmenting the image to define a plurality of single-cell images. A single-cell image may be processed, for example, by applying a filter to the single-cell image to decrease a resolution of the single-cell image as compared to the first resolution, to a second resolution. A label may be assigned to the single-cell image. A machine learning model is trained to predict a classification of the single-cell image based on inputting a plurality of single-cell images into the model. The trained machine learning model may be used to predict the outcome of a treatment. Systems and computer program products are also provided.

### CROSS-REFERENCE TO RELATED APPLICATION

This application claims priority to U.S. Provisional Patent Application No. 63/125,434, filed Dec. 15, 2020, the disclosure of which is incorporated by reference herein in its entirety.

### BACKGROUND

This disclosure relates generally to systems, products, and methods for classification of diseases based on expansion microscopic images and, in non-limiting embodiments or aspects, methods, systems, and computer program products for classification of diseases based on expansion microscopic images.

Histology is a branch of biology that may include the study of tissues, the study of cells, and/or the study of organs. The identification (e.g., using a microscope) and analysis of diseased tissue is a branch of histology called histopathology.

When analyzing an image (e.g., an expanded microscopic image) of a sample (e.g., of a tissue or a cell specimen) at a microscopic level, certain techniques require staining to define major organelle and/or sub-organelles within an individual cell(s). Using existing techniques, it may be challenging to accurately classify a cell type (e.g., normal, cancerous, other, etc.) from a tissue sample.

Therefore, there is a need in the art for an accurate system for classification of cell types.

### SUMMARY

According to a non-limiting embodiment or aspect, provided is a computer-implemented method for classification of diseases based on expansion microscopic images, the method including: receiving, with at least one processor, image data associated with at least one image at a first resolution; removing, with the at least one processor, a background from the at least one image; segmenting, with the at least one processor, the at least one image to define a plurality of single-cell images; applying, with the at least one processor, a filter to at least one single-cell image of the plurality of single-cell images, wherein the filter decreases a resolution of the at least one single-cell image as compared to the first resolution, to a second resolution; assigning, with the at least one processor, a label to the at least one single-cell image of the plurality of single-cell images; and training, with the at least one processor, a machine learning model to predict a classification of the at least one single-cell image of the plurality of single-cell images based on inputting the plurality of single-cell images into the machine learning model.

According to a non-limiting embodiment or aspect, provided is a system for classification of diseases based on expansion microscopic images. The system includes at least one processor. The at least one processor is programmed and/or configured to: receive image data associated with at least one image at a first resolution; remove a background from the at least one image; segment the at least one image to define a plurality of single-cell images; apply a filter to at least one single-cell image of the plurality of single-cell images, wherein the filter decreases a resolution of the at least one single-cell image; assign a label to the at least one single-cell image of the plurality of single-cell images; and train a machine learning model to predict a classification of the at least one single-cell image of the plurality of single-cell images based on inputting the plurality of single-cell images into the machine learning model.

According to a non-limiting embodiment or aspect, provided is a computer program product for classification of diseases based on expansion microscopic images. The computer program product includes at least one non-transitory computer-readable medium including one or more instructions that, when executed by at least one processor, cause the at least one processor to: receive image data associated with at least one image at a first resolution; remove a background from the at least one image; segment the at least one image to define a plurality of single-cell images; apply a filter to at least one single-cell image of the plurality of single-cell images, wherein the filter decreases a resolution of the at least one single-cell image; assign a label to the at least one single-cell image of the plurality of single-cell images; and train a machine learning model to predict a classification of the at least one single-cell image of the plurality of single-cell images based on inputting the plurality of single-cell images into the machine learning model.

According to a non-limiting embodiment or aspect, provided is a computer-implemented method for classifying diseases and predicting the outcome of a treatment based on expansion microscopic images, the method including: receiving, with at least one processor, image data associated with at least one image at a first resolution; removing, with the at least one processor, a background from the at least one image; segmenting, with the at least one processor, the at least one image to define a plurality of single-cell images; inputting, with the at least one processor, at least one single-cell image of the plurality of single-cell images into a trained machine learning model, wherein the trained machine learning model outputs a classification value corresponding to a category of a plurality of categories; sorting, with the at least one processor, the at least one single-cell image of the plurality of single-cell images into one category of the plurality of categories based on the value output by the trained machine learning model; generating, with the at least one processor, a communication including a predicted treatment outcome based on which category of the plurality of categories the at least one single-cell image of the plurality of single-cell images is sorted into; and displaying, with the at least one processor, data associated with the communication via a graphical user interface (GUI) on a user device, wherein the trained machine learning model is trained by: receiving, with at least one processor, image data associated with at least one image at a first resolution; removing, with the at least one processor, a background from the at least one image; segmenting, with the at least one processor, the at least one image to define a plurality of single-cell images; applying, with the at least one processor, a filter to at least one single-cell image of the plurality of single-cell images, wherein the filter decreases a resolution of the at least one single-cell image as compared to the first resolution, to a second resolution; assigning, with the at least one processor, a label to the at least one single-cell image of the plurality of single-cell images; and training, with the at least one processor, a machine learning model to predict a classification of the at least one single-cell image of the plurality of single-cell images based on inputting the plurality of single-cell images into the machine learning model.

According to a non-limiting embodiment or aspect, provided is a system for classifying diseases and predicting the outcome of a treatment based on expansion microscopic images. The system includes at least one processor. The at least one processor is programmed and/or configured to: receive image data associated with at least one image of a plurality of cells at a first resolution; remove a background from the at least one image; deconstruct the at least one image into separate layers; segment the at least one image to define a plurality of single-cell images; input at least one single-cell image of the plurality of single-cell images into a trained machine learning model, wherein the trained machine learning model outputs a classification value corresponding to a category of a plurality of categories; sort the at least one single-cell image of the plurality of single-cell images into one category of the plurality of categories based on the value output by the trained machine learning model; generate a communication including a predicted treatment outcome based on which category of the plurality of categories the at least one single-cell image of the plurality of single-cell images is sorted into; and display data associated with the communication via a graphical user interface (GUI) on a user device.

According to a non-limiting embodiment or aspect, provided is a computer program product for classification of diseases based on expansion microscopic images. The computer program product includes at least one non-transitory computer-readable medium including one or more instructions that, when executed by at least one processor, cause the at least one processor to: receive image data associated with at least one image at a first resolution; remove a background from the at least one image; deconstruct the at least one image into separate layers; segment the at least one image to define a plurality of single-cell images; input at least one single-cell image of the plurality of single-cell images into a trained machine learning model, wherein the trained machine learning model outputs a classification value corresponding to a category of a plurality of categories; sort the at least one single-cell image of the plurality of single-cell images into one category of the plurality of categories based on the value output by the trained machine learning model; generate a communication including a predicted treatment outcome based on which category of the plurality of categories the at least one single-cell image of the plurality of single-cell images is sorted into; and display data associated with the communication via a graphical user interface (GUI) on a user device.

According to a non-limiting embodiment or aspect, provided is a computer-implemented method for analyzing a tissue or cell sample of a patient, the method including: obtaining a tissue or cell sample from the patient; staining the tissue or cell sample with an optically detectable stain for identification of a feature of the tissue or a cell; optionally expanding the stained tissue or cell; obtaining an image of the cell at a wavelength for detection of the staining of the feature of the tissue or cell, wherein the image is at a first resolution; transmitting data of an image to a computer system configured to: remove a background from the image; deconstruct the image into separate layers; segment the image to define a plurality of single-cell images; input at least one single-cell image of the plurality of single-cell images into a trained machine learning model, wherein the trained machine learning model outputs a value corresponding to a category of a plurality of categories; sort the at least one single-cell image of the plurality of single-cell images into one category of the plurality of categories based on the value output by the trained machine learning model; and generate a communication including a predicted treatment outcome based on which category of the plurality of categories the at least one single-cell image of the plurality of single-cell images is sorted into; and displaying data associated with the communication via a graphical user interface (GUI) on a user device. The machine learning model is trained by: receiving, with at least one processor, image data associated with at least one image at a first resolution; removing, with the at least one processor, a background from the at least one image; segmenting, with the at least one processor, the at least one image to define a plurality of single-cell images; applying, with the at least one processor, a filter to at least one single-cell image of the plurality of single-cell images, wherein the filter decreases a resolution of the at least one single-cell image as compared to the first resolution, to a second resolution; assigning, with the at least one processor, a label to the at least one single-cell image of the plurality of single-cell images; and generating, with the at least one processor, a machine learning model to predict a classification of the at least one single-cell image of the plurality of single-cell images based on inputting the plurality of single-cell images into the machine learning model. The method further includes treating the patient according to the data associated with the communication displayed via the GUI on the user device.

Other non-limiting embodiments or aspects are set forth in the following illustrative and exemplary numbered clauses:
Clause 1: A computer implemented method comprising: receiving, with at least one processor, image data associated with at least one image at a first resolution; removing, with the at least one processor, a background from the at least one image; segmenting, with the at least one processor, the at least one image to define a plurality of single-cell images; applying, with the at least one processor, a filter to at least one single-cell image of the plurality of single-cell images, wherein the filter decreases a resolution of the at least one single-cell image as compared to the first resolution, to a second resolution; assigning, with the at least one processor, a label to the at least one single-cell image of the plurality of single-cell images; and training, with the at least one processor, a machine learning model to predict a classification of the at least one single-cell image of the plurality of single-cell images based on inputting the plurality of single-cell images into the machine learning model.
Clause 2: The computer implemented method of clause 1, wherein the at least one image comprises an image of a plurality of cells, and wherein segmenting the at least one image to define the plurality of single-cell images comprises: identifying a location of each cell of the plurality of cells of the at least one image based on a pixel coordinate of each cell of the plurality of cells of the at least one image; and defining for each of the plurality of cells, a single-cell image based on the location of a cell of the plurality of cells, wherein the single-cell image comprises the cell of the plurality of cells and a microenvironment of the cell of the plurality of cells.
Clause 3: The computer implemented method of clauses 1 or 2, wherein the image of the plurality of cells comprises a plurality of nuclei, and wherein prior to identifying the location of each cell of the plurality of cells of the at least one image, the method further comprises: blurring the at least one image by decreasing the resolution of the at least one image to facilitate identification of the plurality of nuclei.
Clause 4: The computer implemented method of any of clauses 1-3, further comprising: repeating, the steps of: applying the filter to the at least one single-cell image of the plurality of single-cell images, wherein the filter increases the resolution of the at least one single-cell image of the plurality of single-cell images as compared to the second resolution and below the first resolution; assigning the label to the at least one single-cell image of the plurality of single-cell images; and training the machine learning model to predict a classification of the at least one single-cell image of the plurality of single-cell images.
Clause 5: The computer implemented method of any of clauses 1-4, further comprising repeating one or more additional times, the steps of: applying the filter to the at least one single-cell image of the plurality of single-cell images; assigning the label to the at least one single-cell image of the plurality of single-cell images; and training the machine learning model to predict a classification of the at least one single-cell image of the plurality of single-cell images, wherein with each subsequent repeat of the steps, the resolution of the filter increases from a previous repetition towards or to the first resolution.
Clause 6: The computer implemented method of any of clauses 1-5, wherein the steps are repeated until the resolution of the at least one single-cell image of the plurality of single-cell images is at the first resolution of the plurality of single-cell images.
Clause 7: The computer implemented method of any of clauses 1-6, wherein the steps are repeated until the resolution of the at least one single-cell image of the plurality of single-cell images is a highest resolution.
Clause 8: The computer implemented method of any of clauses 1-7, wherein the filter is a Gaussian filter.
Clause 9: The computer implemented method of any of clauses 1-8, wherein the machine learning model outputs a classification value for the at least one single-cell image of the plurality of single-cell images, and wherein training the machine learning model to predict a classification of the at least one single-cell image of the plurality of single-cell images further comprises: determining whether an accuracy value of the machine learning model is above a threshold value; based on determining that the accuracy value of the machine learning model is above the threshold value, determining whether the classification predicted for the at least one single-cell image matches a classification of the at least one single-cell image; and based on determining that the classification predicted for the at least one single-cell image does not match a classification of the at least one single-cell image, automatically correcting the classification of the at least one single-cell image to match the classification predicted for the at least one single-cell image.
Clause 10: The computer implemented method of any of clauses 1-9, further comprising: manipulating an image perspective of the at least one single-cell image of the plurality of single-cell images to provide a plurality of augmented single-cell images, wherein manipulating the image perspective comprises randomly flipping, randomly rotating, randomly shearing along an axis, and/or randomly translating at least one single-cell image of the plurality of single-cell images, and/or adding random noise to the at least one single-cell image of the plurality of single-cell images.
Clause 11: The computer implemented method of any of clauses 1-10, wherein the at least one image has a resolution of 5 nm to 250 nm per pixel.
Clause 12: The computer implemented method of any of clauses 1-11, wherein the machine learning model comprises a convolutional neural network (CNN).
Clause 13: The computer implemented method of any of clauses 1-12, wherein the at least one image comprises an image of a physically expanded sample of a tissue or a cell specimen.
Clause 14: The computer implemented method any of clauses 1-13, wherein the sample is expanded by permeating the sample with a polymer monomer composition comprising an α, β-unsaturated carbonyl monomer, such as an acrylate, methacrylate, acrylamide, or methacrylamide monomer for producing a water-swellable (co)polymer, and an enal able to polymerize with the acrylate, methacrylate, acrylamide, or methacrylamide monomer; and polymerizing the polymer monomer composition with the enal to form a swellable material containing the cell or tissue sample, resulting in covalent linking of the enal to both the swellable material and a biomaterial in the sample.
Clause 15: A system comprising at least one processor programmed or configured to: receive image data associated with at least one image at a first resolution; remove a background from the at least one image; segment the at least one image to define a plurality of single-cell images; apply a filter to at least one single-cell image of the plurality of single-cell images, wherein the filter decreases a resolution of the at least one single-cell image; assign a label to the at least one single-cell image of the plurality of single-cell images; and train a machine learning model to predict a classification of the at least one single-cell image of the plurality of single-cell images based on inputting the plurality of single-cell images into the machine learning model.
Clause 16: The system of clause 15, wherein the at least one image comprises an image of a plurality of cells, and wherein when segmenting the at least one image to define the plurality of single-cell images, the at least one processor is further programmed or configured to: identify a location of each cell of the plurality of cells of the at least one image based on a pixel coordinate of each cell of the plurality of cells of the at least one image; and define for each of the plurality of cells, a single-cell image based on the location of a cell of the plurality of cells, wherein the single-cell image comprises the cell of the plurality of cells and a microenvironment of the cell of the plurality of cells.
Clause 17: The system of clauses 15 or 16, wherein the image of the plurality of cells comprises a plurality of nuclei, and wherein prior to identifying the location of each cell of the plurality of cells of the at least one image, the at least one processor is further programmed or configured to: blur the at least one image by decreasing the resolution of the at least one image to facilitate identification of the plurality of nuclei.
Clause 18: The system of any of clauses 15-17, wherein the at least one processor is further programmed or configured to: repeat the steps of: applying the filter to the at least one single-cell image of the plurality of single-cell images, wherein the filter increases the resolution of the at least one single-cell image of the plurality of single-cell images as compared to the second resolution and below the first resolution; assigning the label to the at least one single-cell image of the plurality of single-cell images; and training the machine learning model to predict a classification of the at least one single-cell image of the plurality of single-cell images.
Clause 19: The system of any of clauses 15-18, wherein the at least one processor is further programmed or configured to: repeat one or more additional times, the steps of: applying the filter to the at least one single-cell image of the plurality of single-cell images; assigning the label to the at least one single-cell image of the plurality of single-cell images; and training the machine learning model to predict a classification of the at least one single-cell image of the plurality of single-cell images, wherein with each subsequent repeat of the steps, the resolution of the filter increases from a previous repetition towards or to the first resolution.
Clause 20: The system of any of clauses 15-19, wherein the steps are repeated until the resolution of the at least one single-cell image of the plurality of single-cell images is at the first resolution of the plurality of single-cell images.
Clause 21: The system of any of clauses 15-20, wherein the steps are repeated until the resolution of the at least one single-cell image of the plurality of single-cell images is a highest resolution.
Clause 22: The system of any of clauses 15-21, wherein the filter is a Gaussian filter.
Clause 23: The system of any of clauses 15-22, wherein the machine learning model outputs a classification value for the at least one single-cell image of the plurality of single-cell images, and wherein when training the machine learning model to predict a classification of the at least one single-cell image of the plurality of single-cell images, the at least one processor is further programmed or configured to: determine whether an accuracy value of the machine learning model is above a threshold value; based on determining that the accuracy value of the machine learning model is above the threshold value, determine whether the classification predicted for the at least one single-cell image matches a classification of the at least one single-cell image; and based on determining that the classification predicted for the at least one single-cell image does not match a classification of the at least one single-cell image, automatically correct the classification of the at least one single-cell image to match the classification predicted for the at least one single-cell image.
Clause 24: The system of any of clauses 15-23, wherein the at least one processor is further programmed or configured to: manipulate an image perspective of the at least one single-cell image of the plurality of single-cell images to provide a plurality of augmented single-cell images, wherein manipulating the image perspective comprises randomly flipping, randomly rotating, randomly shearing along an axis, and/or randomly translating at least one single-cell image of the plurality of single-cell images, and/or adding random noise to the at least one single-cell image of the plurality of single-cell images.
Clause 25: The system of any of clauses 15-24, wherein the at least one image has a resolution of 5 nm to 250 nm per pixel.
Clause 26: The system of any of clauses 15-25, wherein the machine learning model comprises a convolutional neural network (CNN).
Clause 27: The system of any of clauses 15-26, wherein the at least one image comprises an image of a physically expanded sample of a tissue or a cell specimen.
Clause 28: The system of any of clauses 15-27, wherein the sample is expanded by permeating the sample with a polymer monomer composition comprising an α, β-unsaturated carbonyl monomer, such as an acrylate, methacrylate, acrylamide, or methacrylamide monomer for producing a water-swellable (co)polymer, and an enal able to polymerize with the acrylate, methacrylate, acrylamide, or methacrylamide monomer; and polymerizing the polymer monomer composition with the enal to form a swellable material containing the cell or tissue sample, resulting in covalent linking of the enal to both the swellable material and a biomaterial in the sample.
Clause 29: A computer program product comprising at least one non-transitory computer readable medium including one or more instructions that, when executed by the at least one processor, cause the at least one processor to: receive image data associated with at least one image at a first resolution; remove a background from the at least one image; segment the at least one image to define a plurality of single-cell images; apply a filter to at least one single-cell image of the plurality of single-cell images, wherein the filter decreases a resolution of the at least one single-cell image; assign a label to the at least one single-cell image of the plurality of single-cell images; and train a machine learning model to predict a classification of the at least one single-cell image of the plurality of single-cell images based on inputting the plurality of single-cell images into the machine learning model.
Clause 30: The computer program product of clause 29, wherein the at least one image comprises an image of a plurality of cells, and wherein the one or more instructions that cause the at least one processor to segment the at least one image to define the plurality of single-cell images further cause the at least one processor to: identify a location of each cell of the plurality of cells of the at least one image based on a pixel coordinate of each cell of the plurality of cells of the at least one image; and define, for each of the plurality of cells, a single-cell image based on the location of a cell of the plurality of cells, wherein the single-cell image comprises the cell of the plurality of cells and a microenvironment of the cell of the plurality of cells.
Clause 31: The computer program product of clauses 29 or 30, wherein the image of the plurality of cells comprises a plurality of nuclei, and wherein prior to identifying the location of each cell of the plurality of cells of the at least one image, the one or more instructions cause the at least one processor to: blur the at least one image by decreasing the resolution of the at least one image to facilitate identification of the plurality of nuclei.
Clause 32: The computer program product of any of clauses 29-31, wherein the one or more instructions cause the at least one processor to: repeat the steps of: applying the filter to the at least one single-cell image of the plurality of single-cell images, wherein the filter increases the resolution of the at least one single-cell image of the plurality of single-cell images as compared to the second resolution and below the first resolution; assigning the label to the at least one single-cell image of the plurality of single-cell images; and training the machine learning model to predict a classification of the at least one single-cell image of the plurality of single-cell images.
Clause 33: The computer program product of any of clauses 29-32, wherein the one or more instructions further cause the at least one processor to repeat one or more additional times, the steps of: applying the filter to the at least one single-cell image of the plurality of single-cell images; assigning the label to the at least one single-cell image of the plurality of single-cell images; and training the machine learning model to predict a classification of the at least one single-cell image of the plurality of single-cell images, wherein with each subsequent repeat of the steps, the resolution of the filter increases from a previous repetition towards or to the first resolution.
Clause 34: The computer program product of any of clauses 29-33, wherein the steps are repeated until the resolution of the at least one single-cell image of the plurality of single-cell images is at the first resolution of the plurality of single-cell images.
Clause 35: The computer program product of any of clauses 29-34, wherein the steps are repeated until the resolution of the at least one single-cell image of the plurality of single-cell images is a highest resolution.
Clause 36: The computer program product of any of clauses 29-35, wherein the filter is a Gaussian filter.
Clause 37: The computer program product of any of clauses 29-36, wherein the machine learning model outputs a classification value for the at least one single-cell image of the plurality of single-cell images, and wherein the one or more instructions that cause the at least one processor to train the machine learning model to predict a classification of the at least one single-cell image of the plurality of single-cell images further cause the at least one processor to: determine whether an accuracy value of the machine learning model is above a threshold value; based on determining that the accuracy value of the machine learning model is above the threshold value, determining whether the classification predicted for the at least one single-cell image matches a classification of the at least one single-cell image; and based on determining that the classification predicted for the at least one single-cell image does not match a classification of the at least one single-cell image, automatically correcting the classification of the at least one single-cell image to match the classification predicted for the at least one single-cell image.
Clause 38: The computer program product of any of clauses 29-37, wherein the one or more instructions further cause the at least one processor to: manipulate an image perspective of the at least one single-cell image of the plurality of single-cell images to provide a plurality of augmented single-cell images, wherein manipulating the image perspective comprises randomly flipping, randomly rotating, randomly shearing an axis, and/or randomly translating at least one single-cell image of the plurality of single-cell images, and/or adding random noise to the at least one single-cell image of the plurality of single-cell images.
Clause 39: The computer program product of any of clauses 29-38, wherein the at least one image has a resolution of 5 nm to 250 nm per pixel.
Clause 40: The computer program product of any of clauses 29-39, wherein the machine learning model comprises a convolutional neural network (CNN).
Clause 41: The computer program product of any of clauses 29-40, wherein the at least one image comprises an image of a physically expanded sample of a tissue or a cell specimen.
Clause 42: The computer program product of any of clauses 29-41, wherein the sample is expanded by permeating the sample with a polymer monomer composition comprising an α, β-unsaturated carbonyl monomer, such as an acrylate, methacrylate, acrylamide, or methacrylamide monomer for producing a water-swellable (co)polymer, and an enal able to polymerize with the acrylate, methacrylate, acrylamide, or methacrylamide monomer; and polymerizing the polymer monomer composition with the enal to form a swellable material containing the cell or tissue sample, resulting in covalent linking of the enal to both the swellable material and a biomaterial in the sample.
Clause 43: A computer implemented method comprising: receiving, with at least one processor, image data associated with at least one image at a first resolution; removing, with the at least one processor, a background from the at least one image; segmenting, with the at least one processor, the at least one image to define a plurality of single-cell images; inputting, with the at least one processor, at least one single-cell image of the plurality of single-cell images into a trained machine learning model, wherein the trained machine learning model outputs a classification value corresponding to a category of a plurality of categories; sorting, with the at least one processor, the at least one single-cell image of the plurality of single-cell images into one category of the plurality of categories based on the value output by the trained machine learning model; generating, with the at least one processor, a communication comprising a predicted treatment outcome based on which category of the plurality of categories the at least one single-cell image of the plurality of single-cell images is sorted into; and displaying, with the at least one processor, data associated with the communication via a graphical user interface (GUI) on a user device, wherein the trained machine learning model is trained by: receiving, with at least one processor, image data associated with at least one image at a first resolution; removing, with the at least one processor, a background from the at least one image; segmenting, with the at least one processor, the at least one image to define a plurality of single-cell images; applying, with the at least one processor, a filter to at least one single-cell image of the plurality of single-cell images, wherein the filter decreases a resolution of the at least one single-cell image as compared to the first resolution, to a second resolution; assigning, with the at least one processor, a label to the at least one single-cell image of the plurality of single-cell images; and training, with the at least one processor, a machine learning model to predict a classification of the at least one single-cell image of the plurality of single-cell images based on inputting the plurality of single-cell images into the machine learning model.
Clause 44: The computer implemented method of clause 43, wherein the image is an image of one or more cells, that are optionally prepared by an expansion microscopy method.
Clause 45: The computer implemented method of clauses 43 or 44, wherein the at least one image comprises an image of a plurality of cells, and wherein segmenting the at least one image to define the plurality of single-cell images comprises: identifying a location of each cell of the plurality of cells of the at least one image based on a pixel coordinate of each cell of the plurality of cells of the at least one image; and defining for each cell of the plurality of cells, at least one single-cell image comprising at least one cell of the plurality of cells and a microenvironment of the at least one cell of the plurality of cells based on the location of the at least one cell of the plurality of cells.
Clause 46: The computer implemented method of any of clauses 43-45, wherein the image of the plurality of cells comprises a plurality of nuclei, and wherein prior to identifying the location of each cell of the plurality of cells of the at least one image, the method further comprises: blurring the at least one image by decreasing the resolution of the at least one image to facilitate identification of the plurality of nuclei.
Clause 47: The computer implemented method of any of clauses 43-46, wherein the at least one image has a resolution of 5 nm to 250 nm per pixel.
Clause 48: The computer implemented method of any of clauses 43-47, wherein the machine learning model comprises a convolutional neural network (CNN).
Clause 49: The computer implemented method of any of clauses 43-48, wherein the plurality of categories comprise normal, cancerous, other, trash, and undetermined.
Clause 50: The computer implemented method of any of clauses 43-49, wherein the at least one image comprises an image of a physically expanded tissue or cell specimen.
Clause 51: The computer implemented method of any of clauses 43-50, wherein the sample is expanded by permeating the sample with a polymer monomer composition comprising an α, β-unsaturated carbonyl monomer, such as an acrylate, methacrylate, acrylamide, or methacrylamide monomer for producing a water-swellable (co)polymer, and an enal able to polymerize with the acrylate, methacrylate, acrylamide, or methacrylamide monomer; and polymerizing the polymer monomer composition with the enal to form a swellable material containing the cell or tissue sample, resulting in covalent linking of the enal to both the swellable material and a biomaterial in the sample.
Clause 52: A system comprising at least one processor programmed or configured to: receive image data associated with at least one image of a plurality of cells at a first resolution; remove a background from the at least one image; deconstruct the at least one image into separate layers; segment the at least one image to define a plurality of single-cell images; input at least one single-cell image of the plurality of single-cell images into a trained machine learning model, wherein the trained machine learning model outputs a classification value corresponding to a category of a plurality of categories; sort the at least one single-cell image of the plurality of single-cell images into one category of the plurality of categories based on the value output by the trained machine learning model; generate a communication comprising a predicted treatment outcome based on which category of the plurality of categories the at least one single-cell image of the plurality of single-cell images is sorted into; and display data associated with the communication via a graphical user interface (GUI) on a user device.
Clause 53: The system of clause 52, wherein the at least one image comprises an image of a plurality of cells, and wherein, when segmenting the at least one image to define the plurality of single-cell images, the at least one processor is further programmed or configured to: identify a location of each cell of the plurality of cells of the at least one image based on a pixel coordinate of each cell of the plurality of cells of the at least one image; and define, for each cell of the plurality of cells, at least one single-cell image comprising at least one cell of the plurality of cells and a microenvironment of the at least one cell of the plurality of cells based on the location of the at least one cell of the plurality of cells.
Clause 54: The system of clauses 52 or 53, wherein the image of the plurality of cells comprises a plurality of nuclei, and wherein prior to identifying the location of each cell of the plurality of cells of the at least one image, the at least one processor is further programmed or configured to: blur the at least one image by decreasing the resolution of the at least one image to facilitate identification of the plurality of nuclei.
Clause 55: The system of any of clauses 52-54, wherein the at least one image has a resolution of 5 nm to 250 nm per pixel.
Clause 56: The system of any of clauses 52-55, wherein the machine learning model comprises a convolutional neural network (CNN).
Clause 57: The system of any of clauses 52-56, wherein the plurality of categories comprise normal, cancerous, other, trash, and undetermined.
Clause 58: The system of any of clauses 52-57, wherein the at least one image comprises an image of a physically expanded tissue or cell specimen.
Clause 59: The system of any of clauses 52-58, wherein the sample is expanded by permeating the sample with a polymer monomer composition comprising an α, β-unsaturated carbonyl monomer, such as an acrylate, methacrylate, acrylamide, or methacrylamide monomer for producing a water-swellable (co)polymer, and an enal able to polymerize with the acrylate, methacrylate, acrylamide, or methacrylamide monomer; and polymerizing the polymer monomer composition with the enal to form a swellable material containing the cell or tissue sample, resulting in covalent linking of the enal to both the swellable material and a biomaterial in the sample.
Clause 60: A computer program product comprising at least one non-transitory computer readable medium including one or more instructions that, when executed by at least one processor, cause the at least one processor to: receive image data associated with at least one image at a first resolution; remove a background from the at least one image; deconstruct the at least one image into separate layers; segment the at least one image to define a plurality of single-cell images; input at least one single-cell image of the plurality of single-cell images into a trained machine learning model, wherein the trained machine learning model outputs a classification value corresponding to a category of a plurality of categories; sort the at least one single-cell image of the plurality of single-cell images into one category of the plurality of categories based on the value output by the trained machine learning model; generate a communication comprising a predicted treatment outcome based on which category of the plurality of categories the at least one single-cell image of the plurality of single-cell images is sorted into; and display data associated with the communication via a graphical user interface (GUI) on a user device.
Clause 61: The computer program product of clause 60, wherein the at least one image comprises an image of a plurality of cells, and wherein the one or more instructions that cause the at least one processor to segment the at least one image to define the plurality of single-cell images further cause the at least one processor to: identify a location of each cell of the plurality of cells of the at least one image based on a pixel coordinate of each cell of the plurality of cells of the at least one image; and define, for each cell of the plurality of cells, at least one single-cell image comprising at least one cell of the plurality of cells and a microenvironment of the at least one cell of the plurality of cells based on the location of the at least one cell of the plurality of cells.
Clause 62: The computer program product of clauses 60 or 61, wherein the image of the plurality of cells comprises a plurality of nuclei, and wherein prior to identifying the location of each cell of the plurality of cells of the at least one image, the one or more instructions further cause the at least one processor to: blur the at least one image by decreasing the resolution of the at least one image to facilitate identification of the plurality of nuclei.
Clause 63: The computer program product of any of clauses 60-62, wherein the at least one image has a resolution of 5 nm to 250 nm per pixel.
Clause 64: The computer program product of any of clauses 60-63, wherein the machine learning model comprises a convolutional neural network (CNN).
Clause 65: The computer program product of any of clauses 60-64, wherein the plurality of categories comprise normal, cancerous, other, trash, and undetermined.
Clause 66: The computer program product of any of clauses 60-65, wherein the at least one image comprises an image of a physically expanded tissue or cell specimen.
Clause 67: The computer program product of any of clauses 60-66, wherein the sample is expanded by permeating the sample with a polymer monomer composition comprising an α, β-unsaturated carbonyl monomer, such as an acrylate, methacrylate, acrylamide, or methacrylamide monomer for producing a water-swellable (co)polymer, and an enal able to polymerize with the acrylate, methacrylate, acrylamide, or methacrylamide monomer; and polymerizing the polymer monomer composition with the enal to form a swellable material containing the cell or tissue sample, resulting in covalent linking of the enal to both the swellable material and a biomaterial in the sample.
Clause 68: A method of analyzing a tissue or cell sample of a patient, comprising: obtaining a tissue or cell sample from the patient; staining the tissue or cell sample with an optically detectable stain for identification of a feature of the tissue or a cell; optionally expanding the stained tissue or cell; obtaining an image of the cell at a wavelength for detection of the staining of the feature of the tissue or cell, wherein the image is at a first resolution; transmitting data of an image to a computer system configured to: remove a background from the image; deconstruct the image into separate layers; segment the image to define a plurality of single-cell images; input at least one single-cell image of the plurality of single-cell images into a trained machine learning model, wherein the trained machine learning model outputs a value corresponding to a category of a plurality of categories; sort the at least one single-cell image of the plurality of single-cell images into one category of the plurality of categories based on the value output by the trained machine learning model; and generate a communication comprising a predicted treatment outcome based on which category of the plurality of categories the at least one single-cell image of the plurality of single-cell images is sorted into; and displaying data associated with the communication via a graphical user interface (GUI) on a user device.
Clause 69: The method of clause 68, wherein the machine learning model is trained by: receiving, with at least one processor, image data associated with at least one image at a first resolution; removing, with the at least one processor, a background from the at least one image; segmenting, with the at least one processor, the at least one image to define a plurality of single-cell images; applying, with the at least one processor, a filter to at least one single-cell image of the plurality of single-cell images, wherein the filter decreases a resolution of the at least one single-cell image as compared to the first resolution, to a second resolution; assigning, with the at least one processor, a label to the at least one single-cell image of the plurality of single-cell images; and generating, with the at least one processor, a machine learning model to predict a classification of the at least one single-cell image of the plurality of single-cell images based on inputting the plurality of single-cell images into the machine learning model.
Clause 70: The method of clauses 68 or 69, further comprising treating the patient according to the data associated with the communication displayed via the GUI on the user device.
Clause 71: The method of clauses 68-70, wherein the image comprises an image of a plurality of cells, and wherein segmenting the image to define the plurality of single-cell images comprises: identifying a location of each cell of the plurality of cells of the image based on a pixel coordinate of each cell of the plurality of cells of the image; and define, for each cell of the plurality of cells, at least one single-cell image comprising at least one cell of the plurality of cells and a microenvironment of the at least one cell of the plurality of cells based on the location of the at least one cell of the plurality of cells.
Clause 72: The method of clauses 68-71, wherein the image of the plurality of cells comprises a plurality of nuclei, and wherein prior to identifying the location of each cell of the plurality of cells of the image, the method further comprises: blurring the image by decreasing the resolution of the image to facilitate identification of the plurality of nuclei.
Clause 73: The method of clauses 68-72, wherein the image has a resolution of 5 nm to 250 nm per pixel.
Clause 74: The method of clauses 68-73, wherein the trained machine learning model comprises a convolutional neural network (CNN).
Clause 75: The method of clauses 68-74, wherein the plurality of categories comprise normal, cancerous, other, trash, and undetermined.
Clause 76: The method of clauses 68-75, wherein optionally expanding the stained tissue or cell comprises: permeating the sample with a polymer monomer composition comprising an α, β-unsaturated carbonyl monomer, such as an acrylate, methacrylate, acrylamide, or methacrylamide monomer for producing a water-swellable (co)polymer, and an enal able to polymerize with the acrylate, methacrylate, acrylamide, or methacrylamide monomer; and polymerizing the polymer monomer composition with the enal to form a swellable material containing the cell or tissue sample, resulting in covalent linking of the enal to both the swellable material and a biomaterial in the sample.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

Additional advantages and details are explained in greater detail below with reference to the non-limiting, exemplary embodiments that are illustrated in the accompanying figures, in which:

FIG. 1 is a diagram of a non-limiting embodiment or aspect of an environment in which systems, devices, products, apparatus, and/or methods, described herein, may be implemented according to the principles of the present disclosure;

FIG. 2 is a diagram of a non-limiting embodiment or aspect of components of one or more devices of FIG. 1;

FIG. 3 is a flow diagram for a non-limiting embodiment or aspect of a process of generating (e.g., training and validating) a machine learning model to classify diseases based on expansion microscopic images;

FIG. 4 is an exemplary flowchart of an implementation of non-limiting embodiments or aspects of the process shown in FIG. 3; and

FIGS. 5-8 are exemplary images of confusion matrices illustrating the training results of a machine learning model generated (e.g., trained and validated) using the process shown in FIG. 3.

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions also refer to word forms, cognates, and grammatical variants of those words or phrases.

For purposes of the description hereinafter, the terms “end,” “upper,” “lower,” “right,” “left,” “vertical,” “horizontal,” “top,” “bottom,” “lateral,” “longitudinal,” and derivatives thereof shall relate to the invention as it is oriented in the drawing figures. However, it is to be understood that the invention may assume various alternative variations and step sequences, except where expressly specified to the contrary. It is also to be understood that the specific devices and processes illustrated in the attached drawings, and described in the following specification, are simply exemplary embodiments or aspects of the invention. Hence, specific dimensions and other physical characteristics related to the embodiments or aspects disclosed herein are not to be considered as limiting.

No aspect, component, element, structure, act, step, function, instruction, and/or the like used herein should be construed as critical or essential unless explicitly described as such. Also, as used herein, the articles “a” and “an” are intended to include one or more items and may be used interchangeably with “one or more” and “at least one.” Furthermore, as used herein, the term “set” is intended to include one or more items (e.g., related items, unrelated items, a combination of related and unrelated items, and/or the like) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise.

As used herein, the terms “comprising,” “comprise” or “comprised,” and variations thereof, in reference to elements of an item, composition, apparatus, method, process, system, claim etc. are intended to be open-ended, meaning that the item, composition, apparatus, method, process, system, claim etc. includes those elements and other elements can be included and still fall within the scope/definition of the described item, composition, apparatus, method, process, system, claim etc. As used herein, “a” or “an” means one or more. As used herein “another” may mean at least a second or more.

As used herein, the term “system” may refer to one or more computing devices or combinations of computing devices such as, but not limited to, processors, servers, client devices, software applications, and/or other like components. In addition, reference to “a server” or “a processor,” as used herein, may refer to a previously-recited server and/or processor that is recited as performing a previous step or function, a different server and/or processor, and/or a combination of servers and/or processors. For example, as used in the specification and the claims, a first server and/or a first processor that is recited as performing a first step or function may refer to the same or different server and/or a processor recited as performing a second step or function.

As used herein, the term “computing device” may refer to one or more electronic devices configured to process data. A computing device may include the necessary components to receive, process, and output data, such as a processor, a display, a memory, an input device, a network interface, and/or the like. A computing device may be a mobile device, such as a cellular phone (e.g., a smartphone), a portable computer, a wearable device (e.g., watches, glasses, lenses, clothing, and/or the like), a personal digital assistant (PDA), and/or other like devices. A computing device may also include a desktop computer or other form of non-mobile computer.

As used herein, the terms “patient” or “subject” refer to members of the animal kingdom, including, but not limited to human beings.

As used herein, the terms “communication” and “communicate” refer to the receipt or transfer of one or more signals, messages, commands, or other type of data. For one unit (e.g., any device, system, or component thereof) to be in communication with another unit means that the one unit is able to directly or indirectly receive data from and/or transmit data to the other unit. This may refer to a direct or indirect connection that is wired and/or wireless in nature. Additionally, two units may be in communication with each other even though the data transmitted may be modified, processed, relayed, and/or routed between the first and second unit. For example, a first unit may be in communication with a second unit even though the first unit passively receives data and does not actively transmit data to the second unit. As another example, a first unit may be in communication with a second unit if an intermediary unit processes data from one unit and transmits processed data to the second unit. It will be appreciated that numerous other arrangements are possible.

As used herein, the term “computing device” or “computer” may refer to one or more electronic devices configured to process data. A computing device may, in some examples, include the necessary components to receive, process, and output data, such as a display, a processor, a memory, an input device, and a network interface. A computing device may be a server, a mobile device, a desktop computer, a subsystem or integrated part of a genomic sequencer or sequence analyzer, and/or the like. As an example, a mobile device may include a cellular phone (e.g., a smartphone or standard cellular phone), a portable computer, a wearable device (e.g., watches, glasses, lenses, clothing, and/or the like), a personal digital assistant (PDA), and/or other like devices.

As used herein, “interface” refers, in the context of programming and software modules, to the languages, codes and messages that programs or modules use to communicate with each other and to the hardware, and includes computer code or other data stored on a computer-readable medium that may be executed by a processor to facilitate the interaction between software modules. In some aspects of the methods and systems described herein, software modules, such as the variant calling module, the tumor phylogeny or modules and the machine learning modules are designed as separate software components, modules, or engines, with each requiring specific data input formats, and providing specific data output formats, and, in non-limiting examples, an interface may be used to facilitate such communication between components.

As used herein, the term “graphical user interface” or “GUI” refers to a generated display with which a user may interact, either directly or indirectly (e.g., through a keyboard, mouse, touchscreen, and/or the like).

As used herein, the term “satisfying” with respect to a threshold may include meeting and/or exceeding a threshold, which may include meeting or having a value less than a minimum-type threshold, and meeting or having a value greater than a maximum-type threshold.

As used herein, the terms “medical treatment” or “treatment,” with respect to a patient, refers to taking one or more actions to improve the current and/or future condition of the patient. Medical treatment may include, but is not limited to, one or more of the following actions: administering a medication or other aid (e.g., oxygen) to the patient, modifying a level of monitoring of the patient, conducting one or more tests of the patient, conducting one or more surgical or reparative operations on the patient, providing one or more therapies or therapeutics to the patient, employing one or more medical devices for use on, in, or by the patient, modifying the position of the patient, increasing or reducing patient stimulation, modifying a diet of the patient, modifying an environment of the patient, and/or the like.

Specific labels and designations are intended to be illustrative, and include, for example and without limitation: synonyms, abbreviations, acronyms, and translations thereof. For example, reference to the labels “normal, cancerous, other, trash, and undetermined” includes like designations, including, e.g., synonyms, abbreviations, acronyms, and translations thereof.

Provided herein are methods, systems, and computer program products that are useful for analysis of micrographic images, such as expanded (e.g., by expansion microscopy method(s), as described herein) photomicrograph images. Small structures (e.g., biomolecules, proteins, DNA, and/or RNA) within fixed cells and tissues are often too small for successful optical microscopic imaging. Expansion microscopy enables super-resolution optical interrogations and overcomes the optical diffraction limit of conventional optical microscopy. Expansion microscopy was developed to allow for the imaging of thick, preserved specimens with approximately 70 nanometers (nm) lateral resolution. With expansion microscopy, a biological specimen may be expanded prior to imaging, bringing previously sub-diffraction limited structures to a size within the range of a conventional microscope with nanoscale precision. For example and without limitation, a technician may introduce a polymer network into cellular or tissue samples, and then physically expand that polymer network using chemical reactions to increase the size of the biological structures. Among other benefits, expansion microscopy allows those small structures to be imaged with a wider range of microscopy techniques. Expanded photomicrographic images as described in, for example and without limitation, International Patent Application Publication Nos. WO 2015/127183, WO 2017/027368, WO 2017/027367, WO 2017/147435, and WO 2019/241662, and in Zhao et al., Nanoscale imaging of clinical specimens using pathology-optimized expansion microscopy. Nat Biotechnol. 2017 Aug.; 35(8):757-764, each of which is incorporated herein by reference in its entirety.

An opportunity follows from expansion microscopy techniques to automate analysis and classification of images of an expanded cell or tissue. For example, a system may be desired that automates classification of tissue or cells as diseased as compared to normal tissue or cells. As described herein, it has been found that straight-forward image analysis, will not suffice for machine learning methods of classification of cells in an expansion microscopy image. As further described herein, system training methods that employ blurring filters, starting with a blurred image, and employing a series of blurring filters with step-wise increased resolution, result in increased system classification accuracy. It is thought, without any intention of being held by this theory, that because microscopy, especially expansion microscopy, increases sub-cellular detail, those details confuse the machine-learning systems, resulting in lower classification accuracy.

Non-limiting embodiments or aspects of the present disclosure are directed to methods, systems, and/or products for classification of diseases based on expansion microscopic images. In some non-limiting embodiments or aspects, a method may include receiving image data associated with at least one image at a first resolution, wherein the image data includes one or more color channels; removing a background from the at least one image; deconstructing the at least one image into separate layers based on each of the one or more color channels for the at least one image; segmenting the at least one image to define a plurality of single-cell images (e.g., a plurality of images of a singular cell including the major organelle and sub-organelle structure of a cell); applying a filter to at least one single-cell image of the plurality of single-cell images wherein the filter decreases a resolution of the at least one single-cell image as compared to the first resolution, to a second resolution; assigning a label to the at least one single-cell image of the plurality of single-cell images; and training a machine learning model to predict a classification of the at least one single-cell image of the plurality of single-cell images based on inputting the plurality of single-cell images into the machine learning model. In some non-limiting embodiments or aspects, the at least one image has a resolution of 5 nm to 250 nm per pixel. In some non-limiting embodiments or aspects, the machine learning model includes, without limitation, a deep learning network or a deep-learning architecture such as a deep neural network, a deep belief network, deep reinforcement learning, a recurrent neural networks, or a convolutional neural network. The Example below, describes one example of a useful machine learning network. Other non-limiting examples of machine learning networks include GoogLeNet, AlexNet, Xception, among others, and such networks may be utilized in the methods, systems, and computer program products, as described herein. A deep neural network may be an artificial neural network with multiple layers between the input and output layers.

In some non-limiting embodiments or aspects, the at least one image may include an image of a plurality of cells. In some non-limiting embodiments or aspects, segmenting the at least one image to define the plurality of single-cell images includes: identifying a location of each cell of the plurality of cells of the a least one image based on a pixel coordinate of each cell of the plurality of cells of the at least one images; and defining a single-cell image based on the location of a cell of the plurality of cells, wherein the single-cell images include the cell of the plurality of cells and a microenvironment of the cell of the plurality of cells. In some non-limiting embodiments or aspects, the image of the plurality of cells includes a plurality of nuclei and prior to identifying the location of each cell of the plurality of cells of the at least one image, the method includes blurring the at least one image by decreasing the resolution of the at least one image to facilitate identification of the plurality of nuclei.

In some non-limiting embodiments or aspects, described systems, methods, and/or products provide for repeating the steps of: applying the filter to the at least one single-cell image of the plurality of single-cell images, wherein the filter increases the resolution of the at least one single-cell image of the plurality of single-cell images as compared to the second resolution and below the first resolution; assigning the label to the at least one single-cell image of the plurality of single-cell images; and training the machine learning model to predict a classification of the at least one single-cell image of the plurality of single-cell images. In some non-limiting embodiments or aspects, the steps are repeated one or more additional times, wherein with each subsequent repeat of the steps, the resolution of the filter increases from a previous repetition towards or to the first resolution. In some non-limiting embodiments or aspects, the steps are repeated until the resolution of the at least one single-cell image of the plurality of single-cell images is at the first resolution of the plurality of single-cell images. In some non-limiting embodiments or aspects, the steps are repeated until the resolution of the at least one single-cell image of the plurality of single-cell images is a highest resolution. In some non-limiting embodiments or aspects, the filter is a Gaussian filter.

In some non-limiting embodiments or aspects, the machine learning model outputs a classification value corresponding to a classification predicted for the at least one single-cell image of the plurality of single-cell images. In some non-limiting embodiments or aspects, training the machine learning model to predict a classification of the at least one single-cell image of the plurality of single-cell images includes: determining whether an accuracy value of the machine learning model is above a threshold value; based on determining that the accuracy value of the machine learning model is above the threshold value, determining whether the classification predicted for the at least one single-cell image matches a classification of the at least one single-cell image; and based on determining that the classification predicted for the at least one single-cell image does not match a classification of the at least one single-cell image, automatically correcting a classification of the at least one single-cell image to match the classification predicted for the at least one single-cell image.

In some non-limiting embodiments or aspects, described systems, methods, and/or products provide for manipulating an image perspective of the at least one single-cell of the plurality of single-cell images to provide a plurality of augmented single-cell images. In some non-limiting embodiments or aspects, manipulating the image perspective includes: randomly flipping, randomly rotating, randomly shearing along an axis, and/or randomly translating at least one single-cell image of the plurality of single-cell images, and/or adding random noise to the at least one single-cell image of the plurality of single-cell images.

In some non-limiting embodiments or aspects, the at least one image includes an image of a physically expanded sample of a tissue of a cell specimen. In some non-limiting embodiments or aspects, the sample is expanded by permeating the sample with a polymer monomer composition comprising an α, β-unsaturated carbonyl monomer, such as an acrylate, methacrylate, acrylamide, or methacrylamide monomer for producing a water-swellable (co)polymer, and an enal able to polymerize with the acrylate, methacrylate, acrylamide, or methacrylamide monomer; and polymerizing the polymer monomer composition with the enal to form a swellable material containing the cell or tissue sample, resulting in covalent linking of the enal to both the swellable material and a biomaterial in the sample.

Additionally, in some non-limiting embodiments or aspects, described systems, methods, and/or products provide for receiving image data associated with at least one image at a first resolution; removing a background from the at least one image; deconstructing the at least one image into separate layers; segmenting the at least one image to define a plurality of single-cell images; inputting at least one single-cell image of the plurality of single-cell images into a trained machine learning model, wherein the trained machine learning model outputs a classification value corresponding to a category of a plurality of categories; sorting the at least one single-cell image of the plurality of single-cell images into one category of the plurality of categories based on the value output by the trained machine learning model; generating a communication comprising a predicted treatment outcome based on which category of the plurality of categories the at least one single-cell image of the plurality of single-cell images is sorted into; and displaying data associated with the communication via a graphical user interface (GUI) on a user device. In some non-limiting embodiments or aspects, the trained machine learning model may be trained using systems or methods as described herein.

Additionally, in some non-limiting embodiments or aspects, described systems, methods, and/or products provide for analyzing a tissue or cell sampled of a patient by: obtaining a tissue or cell sample from the patient; staining the tissue or cell sample with an optically detectable stain for identification of a feature of the tissue or a cell; optionally expanding the stained tissue or cell; obtaining an image of the cell at a wavelength for detection of the staining of the feature of the tissue or cell, wherein the image is at a first resolution; transmitting data of the image to a remote computer system, receiving, by a user device, the communication comprising the predicted treatment outcome from the remote computer system; and displaying data associated with the communication via a GUI on the user device. In some non-limiting embodiments or aspects, in response to receiving the data of the image, the remote computer system: removes a background from the image, deconstructs the image into separate layers, segments the image to define a plurality of single-cell images, inputs at least one single-cell image of the plurality of single-cell images into a trained machine learning model, wherein the trained machine learning model outputs a classification value corresponding to a category of a plurality of categories, sorts the at least one single-cell image of the plurality of single-cell images into one category of the plurality of categories based on the value output by the trained machine learning model, and generates a communication comprising a predicted treatment outcome based on which category of the plurality of categories the at least one single-cell image of the plurality of single-cell images is sorted into.

In this way, non-limiting embodiments or aspects of the present disclosure improve the accuracy of classifying cell types that are challenging to human pathologists. In some non-limiting embodiments or aspects, described systems, methods, and/or products can extract more (e.g., 2-3 orders of magnitude more) information from nanoscopic images than traditional methods and/or systems. Further, non-limiting embodiments or aspects of the present disclosure improve training of the machine learning model by forcing the model to lean low resolution information first before focusing on nano-scale details, thereby increasing the classification accuracy and avoiding the problem of having the model stuck in the local optimal.

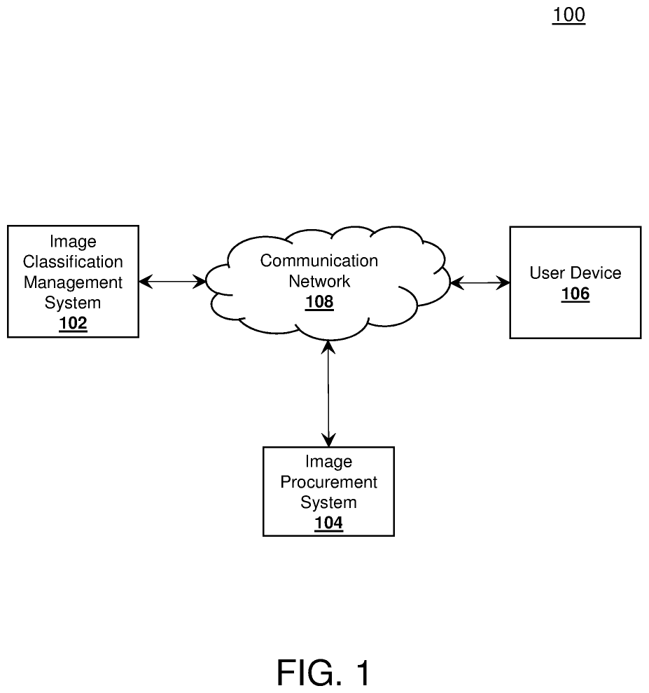

Referring now to FIG. 1, shown is a diagram of a non-limiting embodiment or aspects of an environment 100 in which devices, systems, and/or methods, described herein, may be implemented. As shown in FIG. 1, environment 100 includes image classification management system 102, image procurement system 104, user device 106, and communication network 108. Image classification management system 102, image procurement system 104, and/or user device 106 may interconnect (e.g., establish a connection to communicate) via wired connections, wireless connections, or a combination of wired and wireless connections.

Image classification management system 102 may include one or more devices configured to communicate with image procurement system 104 and/or user device 106 via communication network 108. For example, image classification management system 102 may include a server, a group of servers, and/or other like devices. Additionally or alternatively, image classification management system 102 may generate (e.g., train, validate, retrain, and/or the like), store, and/or implement (e.g., operate, provide inputs to and/or outputs from, and/or the like) one or more machine learning models. In some non-limiting embodiments or aspects, image classification management system 102 may be in communication with a data storage device, which may be local to (e.g., a component of) or remote from (e.g., a component of a device or system that is in communication with) image classification management system 102. In some non-limiting embodiments or aspects, image classification management system 102 may be capable of receiving information from, storing information in, transmitting information to, and/or searching information stored in the data storage device.

Image procurement system 104 may include one or more devices configured to communicate with image classification management system 102 and/or user device 106 via communication network 108. For example, image procurement system 104 may include a computing device, such as a server, a group of servers, and/or other like devices. In some non-limiting embodiments, image procurement system 104 may include a microscope, such as an electron microscope or an optical microscope, which is capable of producing images on a microscopic and/or nanoscopic scale. In some non-limiting embodiments or aspects, image classification management system 102 may be a component of image procurement system 104, or vice versa.

User device 106 may include a computing device configured to communicate with image classification management system 102 and/or image procurement system 104 via communication network 108. For example, user device 106 may include a computing device, such as a desktop computer, a portable computer (e.g., tablet computer, a laptop computer, and/or the like), a mobile device (e.g., a cellular phone, a smartphone, a personal digital assistant, a wearable device, and/or the like), and/or other like devices. In some non-limiting embodiments or aspects, user device 106 may be associated with a user (e.g., an individual operating user device 106). In some non-limiting embodiments or aspects, user device 106 may be a component of image procurement system 104.

Communication network 108 may include one or more wired and/or wireless networks. For example, communication network 108 may include a cellular network (e.g., a long-term evolution (LTE) network, a third generation (3G) network, a fourth generation (4G) network, a fifth generation (5G) network, a code division multiple access (CDMA) network, etc.), a public land mobile network (PLMN), a local area network (LAN), a wide area network (WAN), a metropolitan area network (MAN), a telephone network (e.g., the public switched telephone network (PSTN) and/or the like), a private network, an ad hoc network, an intranet, the Internet, a fiber optic-based network, a cloud computing network, and/or the like, and/or a combination of some or all of these or other types of networks.

The number and arrangement of devices and networks shown in FIG. 1 are provided as an example. There may be additional devices and/or networks, fewer devices and/or networks, different devices and/or networks, or differently arranged devices and/or networks than those shown in FIG. 1. Furthermore, two or more devices shown in FIG. 1 may be implemented within a single device, or a single device shown in FIG. 1 may be implemented as multiple, distributed devices. Additionally or alternatively, a set of devices (e.g., one or more devices) of environment 100 may perform one or more functions described as being performed by another set of devices of environment 100.

With continued reference to FIG. 1, in some non-limiting embodiments or aspects, image classification management system 102 may receive (e.g., from image procurement system 104) image data, as described herein. For example, image classification management system 102 may receive image data associated with at least one image from image procurement system 104. The image data may be nanoscale image data. The image data may be associated with at least one image at a first resolution. For example, the at least one image may have a resolution of 5 nm to 250 nm per pixel. The image data may include one or more color channels (e.g., RGB image has three color channels: red, green, and blue; CMYK image has four color channels: cyan, magenta, yellow, and key). The color channels may correspond to a range of wavelengths and contain spectroscopic information.

In some non-limiting embodiments or aspects, the at least one image may include an image of a physically expanded sample of a tissue or a cell specimen. In some non-limiting embodiments or aspects, the sample may be expanded by permeating the sample with a polymer monomer composition comprising an α, β-unsaturated carbonyl monomer, such as an acrylate, methacrylate, acrylamide, or methacrylamide monomer for producing a water-swellable (co)polymer, and an enal able to polymerize with the acrylate, methacrylate, acrylamide, or methacrylamide monomer; and polymerizing the polymer monomer composition with the enal to form a swellable material containing the cell or tissue sample, resulting in covalent linking of the enal to both the swellable material and a biomaterial in the sample.

The images processed according to the present disclosure may be images of one cell, or a plurality of cells. The image (e.g., a photomicrograph) may be taken using any suitable microscope, such as an optical or digital microscope, including photomicrography, fluorescence photomicrography, confocal photomicrography, and/or the like. Images are typically taken in the visible light range (e.g., from 380 nm to 700 nm), but may range more broadly into the ultraviolet (UV) (e.g. from 10 nm to 400 nm) or infrared (e.g., near infrared, from 800 nm to 2,500 nm) ranges. Cellular, sub-cellular, and/or extracellular structures may be dyed using a colored dye or a fluorescent dye to assist in visualization and, more pertinently in the context of the present invention, to distinguish such cellular, sub-cellular, and/or extracellular structures for the purpose of analysis according to the invention described herein. Suitable dyes, or combinations of dyes (e.g., 1, 2, 3, 4, or more different dyes) may be selected to distinguish a cell to be analyzed from a normal cell. Suitable dyes and dying techniques are broadly-known to persons of ordinary skill in the photomicrography arts. Illustrative examples of colored dyes include H&E (hematoxylin and eosin), DAPI (4′, 6-diamidino-2-phenylindole, which stains DNA), Nile blue, ethidium bromide, Hoechst stains, lysosomal stains, and the like. As is broadly-known, binding reagents, such as antibodies, antibody fragments, antibody-based binding reagents, nucleic acids (e.g., for fluorescence in situ hybridization, or FISH), aptamers, lectins, receptors, agonists, metabolites, etc. may be conjugated with a dye, such as a fluorescent dye, to specifically-label nucleic acids, proteins, polysaccharides, lipids, or any other cellular component. Useful specific and non-specific dye reagents are broadly commercially-available and/or are prepared using common techniques. Suitable fluorescent dyes that are conjugated to specific binding reagents, such as antibodies include, for example and without limitation: rhodamines, cyanines, xanthenes (e.g., fluorescein, or FITC), among many others. Imaging of cells and cell fluorescence may be accomplished by any useful method, which are broadly-known. For example H&E staining may be imaged using visible light, while fluorescence may be imaged at one or more emission wavelengths for the fluorophores used, and illuminated at one or more excitation wavelengths for the fluorophores used. In the Example below, and as non-limiting examples of suitable stain combinations, in a first training set bladder biopsies were stained with DAPI and FISH-targeting the Centromere protein B (CENPB) box, and in another training set using a bladder biopsy, DAPI, FISH-CENPB, WGA (wheat germ agglutinin), and miR140-5p (microRNA140-5p) were differentially-stained, both yielding excellent classification of the cells (>99% training accuracy).

Additionally or alternatively, image classification management system 102 may remove (e.g., subtract) a background from the at least one image, as described herein. Removing a background may include comparing the at least one image to a control image, wherein the control image does not include any cells or tissue; comparing the at least one image to a portion of the at least one image with no cells; and/or performing image manipulations. Image manipulations to remove a background from at least one image may include, without limitations, adjusting a contrast of the image, adjusting a brightness of the image, adjusting color saturation of the image, and/or any other useful method of removing non-specific or undesirable information from the image.

Additionally or alternatively, image classification management system 102 may deconstruct the at least one image into separate layers, as described herein. For example, image classification management system 102 may deconstruct the at least one image into separate layers based on each of the one or more color channels of the at least one image. In some non-limiting embodiments or aspects, the number of layers may correspond to the number of color channels of the at least one image.

Additionally or alternatively, image classification management system 102 may segment the at least one image to define a plurality of single-cell images, as described herein. The at least one image may include an image of a plurality of cells. In some non-limiting embodiments or aspects, when segmenting the at least one image to define the plurality of images, image classification management system 102 may identifying a location of each cell of the plurality of cells of the at least one image, as described herein. For example, image classification management system 102 may identify a location of each cell of the plurality of cells of the at least one image based on a pixel coordinate of each cell of the plurality of cells of the at least one image. In some non-limiting embodiments or aspects, image classification management system 102 may define, for each of the plurality of cells, a single-cell image. For example, image classification management system 102 may define, for each of the plurality of cells, a single-cell image based on the location of a cell of the plurality of cells. The single-cell image may include the cell of the plurality of cells and/or a microenvironment of the cell of the plurality of cells. The image of the plurality of cells may include a plurality of nuclei. In some non-limiting embodiments or aspects, prior to identifying the location of each cell of the plurality of cells of the at least one image, image classification management system 102 may blur the at last one image. For example, image classification management system 102 may decrease a resolution of the at least one image causing the at least one image to blur, soften, smooth, and/or pixelate in order to facilitate identification of the plurality of nuclei. Decreasing the resolution may exclude sub-organelle information of the nuclei in the at least one image such that major organelle (e.g., the nucleus of a cell) can be identified and/or distinguished but sub-structures thereof cannot be identified and/or distinguished. Image classification management system 102 may apply a filter to the at least one image to decrease the resolution of the at least one image.

Additionally or alternatively, image classification management system 102 may apply a filter to at least one single-cell image of the plurality of single-cell images, as described herein. The filter may decrease a resolution of the at least one single-cell image as compared to the first resolution, to a second resolution. For example, image classification management system 102 may apply a filter (e.g., image signal processing filter(s)) to at least one single-cell image of the plurality of single-cell images to decrease the resolution of the at least one single-cell image from a first resolution to a second resolution. In some non-limiting embodiments or aspects, the filter may be a Gaussian filter.

Additionally or alternatively, image classification management system 102 may assign a label to the at least one single-cell image of the plurality of single-cell images, as described herein. In some non-limiting embodiments or aspects, image data associated with the at least one image may include a label. For example, the at least one image may include an image from a labeled region of interest of a patient's sample. In some non-limiting embodiments or aspects, a label may include: normal, cancerous, other, trash, undetermined, training data, validation data, test data, a disease name, a category of a disease, a sub-category of a disease, and/or the like.

Additionally or alternatively, image classification management system 102 may train a machine learning (ML) model to predict a classification of the at least one single-cell image of the plurality of single-cell images, as described herein. For example, the image classification management system 102 may train a ML model to perform a task (e.g., predict a classification of the at least one single-cell image of the plurality of single-cell images) based on inputting the plurality of single-cell images into a machine learning model. The ML model may be a supervised ML model (e.g., a ML model using labeled data). In some non-limiting embodiments or aspects, the ML model may include a convolutional neural network (CNN). Additionally or alternatively, in some non-limiting embodiments or aspects, the ML model may include a residual network (ResNet). In some non-limiting embodiments or aspects, the ML model may output a classification value corresponding to a classification predicted for the at least one single-cell image of the plurality of single-cell images. For example, ML image classification management system 102 may output a classification value corresponding to a classification predicted for the at least one single-cell image of the plurality of single-cell images. In some non-limiting embodiments or aspects, training the machine learning model to predict a classification of the at least one single-cell image of the plurality of single-cell images may include: determining whether an accuracy value of the machine learning model is above a threshold value; based on determining that the accuracy value of the machine learning model is above the threshold value, determining whether the classification predicted for the at least one single-cell image matches a classification of the at least one single-cell image; and based on determining that the predicted label for the at least one single-cell image does not match a classification of the at least one single-cell image, automatically correcting a classification of the at least one single-cell image to match the classification predicted for the at least one single-cell image.

Additionally or alternatively, image classification management system 102 may repeat the steps of applying the filter to the at least one single-cell image of the plurality of single-cell images, wherein the filter increases the resolution of the at least one single-cell image of the plurality of single-cell images as compared to the second resolution and below the first resolution; assigning the label to the at least one single-cell image of the plurality of single-cell images; and training the machine learning model to predict a classification of the at least one single-cell image of the plurality of single-cell images, as described herein. The steps may be repeated one or more additional times. In some non-limiting embodiments or aspects, with each subsequent repeat of the steps, the resolution of the filter may increase from a previous repetition towards or to the first resolution. In some non-limiting embodiments or aspects, the steps may be repeated until the resolution of the at least one single-cell image of the plurality of single-cell images is at the first resolution of the plurality of single-cell images. In some non-limiting embodiments or aspects, the steps may be repeated until the resolution of the at least one single-cell image of the plurality of single-cell images is a highest resolution.

Additionally or alternatively, prior to training the ML model, image classification management system 102 may manipulate an image perspective of the at least one single-cell image of the plurality of single-cell images to provide a plurality of augmented single-cell images, as described herein. For example, image classification management system 102 may manipulate the image perspective by randomly flipping, randomly rotating, randomly shearing along an axis, and/or randomly translating at least one single-cell image of the plurality of single-cell images, and/or adding random noise to the at least one single-cell image of the plurality of single-cell images.

Additionally or alternatively, image classification management system 102 may receive image data associated with at least one image at a first resolution; remove a background from the at least one image; deconstruct the at least one image into separate layers; segment the at least one image to define a plurality of single-cell images; and input at least one single-cell image of the plurality of single-cell images into a trained ML model, as described herein. For example, image classification management system 102 may receive image data associated with at least one image at a first resolution; remove a background from the at least one image; deconstruct the at least one image into separate layers; segment the at least one image to define a plurality of single-cell images; and input at least one single-cell image into ML model system 106. In some non-limiting embodiments or aspects, the trained ML model is trained using systems, methods, and/or products as described herein. In some non-limiting embodiments or aspects, the trained ML model may output a classification value corresponding to a category of a plurality of categories. For example, image classification management system 102 may output a classification value corresponding to a category of a plurality of categories. The plurality of categories may include: include: normal, cancerous, other, trash, undetermined, training data, validation data, test data, a disease name, a category of a disease, a sub-category of a disease, and/or the like.

Additionally or alternatively, image classification management system 102 may sort the at least one single-cell image of the plurality of single-cell images into one category of the plurality of categories, as described herein. For example, image classification management system 102 may sort the at least one single-cell image of the plurality of single-cell images into one category of the plurality of categories based on the value output by the trained ML model.

Additionally or alternatively, image classification management system 102 may generate a communication, as described herein. For example, image classification management system 102 may generate a communication comprising a predicted treatment outcome based on which category of the plurality of categories the at least one single-cell image of the plurality of single-cell images is sorted into. The communication may be a notification and/or a customized message (e.g., customized based on image data and/or patient data).

Additionally or alternatively, image classification management system 102 may display data associated with the communication via a graphical user interface (GUI), as described herein. For example, image classification management system 102 may display data associated with the communication system via a GUI on user device 106. The GUI may be an interactive GUI. In some non-limiting embodiments or aspects, the interactive GUI may comprise at least one selectable option and/or at least one input option. The interactive GUI may be configured to be updated based on receiving the at least one selection from a user and/or the at least one input from a user.

The number and arrangement of systems and databases shown in FIG. 1 are provided as an example. There may be additional systems and/or databases, fewer systems and/or databases, different systems and/or databases, and/or differently arranged systems and/or databases than those shown in FIG. 1. Furthermore, two or more systems shown in FIG. 1 may be implemented within a single system, or a single system or a single database shown in FIG. 1 may be implemented as multiple, distributed systems or databases. Additionally, or alternatively, a set of systems or a set of databases (e.g., one or more systems, one or more databases, etc.) of environment 100 may perform one or more functions described as being performed by another set of systems or another set of databases of environment 100.

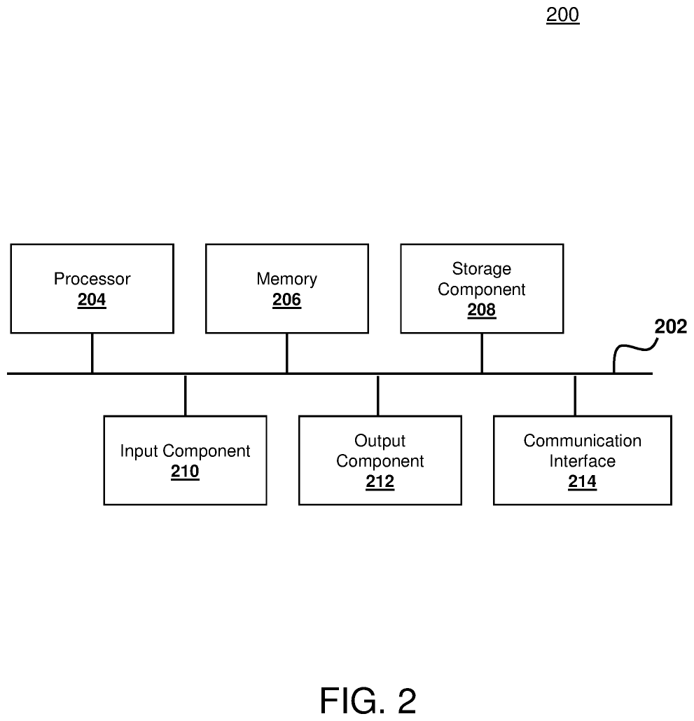

Referring now to FIG. 2, shown is a diagram of example components of a device 200. Device 200 may correspond to image classification management system 102 (e.g., one or more devices of image classification management system 102), image procurement system 104 (e.g., one or more devices of image procurement system 104), and/or user device 106. In some non-limiting embodiments or aspects, image classification management system 102, image procurement system 104, and/or user device 106 may include at least one device 200 and/or at least one component of device 200. As shown in FIG. 2, device 200 may include bus 202, processor 204, memory 206, storage component 208, input component 210, output component 212, and communication interface 214.

Bus 202 may include a component that permits communication among the components of device 200. In some non-limiting embodiments or aspects, processor 204 may be implemented in hardware, software, or a combination of hardware and software. For example, processor 204 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 206 may include random access memory (RAM), read-only memory (ROM), and/or another type of dynamic or static storage memory (e.g., flash memory, magnetic memory, optical memory, etc.) that stores information and/or instructions for use by processor 204.

Storage component 208 may store information and/or software related to the operation and use of device 200. For example, storage component 208 may include a hard disk (e.g., a magnetic disk, an optical disk, a magneto-optic disk, a solid state disk, etc.), a compact disc (CD), a digital versatile disc (DVD), a floppy disk, a cartridge, a magnetic tape, and/or another type of computer-readable medium, along with a corresponding drive.

Input component 210 may include a component that permits device 200 to receive information, such as via user input (e.g., a touch screen display, a keyboard, a keypad, a mouse, a button, a switch, a microphone, etc.). Additionally or alternatively, input component 210 may include a sensor for sensing information (e.g., a global positioning system (GPS) component, an accelerometer, a gyroscope, an actuator, etc.). Output component 212 may include a component that provides output information from device 200 (e.g., a display, a speaker, one or more light-emitting diodes (LEDs), etc.).

Communication interface 214 may include a transceiver-like component (e.g., a transceiver, a separate receiver and transmitter, etc.) that enables device 200 to communicate with other devices, such as via a wired connection, a wireless connection, or a combination of wired and wireless connections. Communication interface 214 may permit device 200 to receive information from another device and/or provide information to another device. For example, communication interface 214 may include an Ethernet interface, an optical interface, a coaxial interface, an infrared interface, a radio frequency (RF) interface, a universal serial bus (USB) interface, a Wi-Fi® interface, a cellular network interface, and/or the like.

Device 200 may perform one or more processes described herein. Device 200 may perform these processes based on processor 204 executing software instructions stored by a computer-readable medium, such as memory 206 and/or storage component 208. A computer-readable medium (e.g., a non-transitory computer-readable medium) is defined herein as a non-transitory memory device. A non-transitory memory device includes memory space located inside of a single physical storage device or memory space spread across multiple physical storage devices.

Software instructions may be read into memory 206 and/or storage component 208 from another computer-readable medium or from another device via communication interface 214. When executed, software instructions stored in memory 206 and/or storage component 208 may cause processor 204 to perform one or more processes described herein. Additionally or alternatively, hardwired circuitry may be used in place of or in combination with software instructions to perform one or more processes described herein. Thus, embodiments or aspects described herein are not limited to any specific combination of hardware circuitry and software.

The number and arrangement of components shown in FIG. 2 are provided as an example. In some non-limiting embodiments or aspects, device 200 may include additional components, fewer components, different components, or differently arranged components than those shown in FIG. 2. Additionally or alternatively, a set of components (e.g., one or more components) of device 200 may perform one or more functions described as being performed by another set of components of device 200.

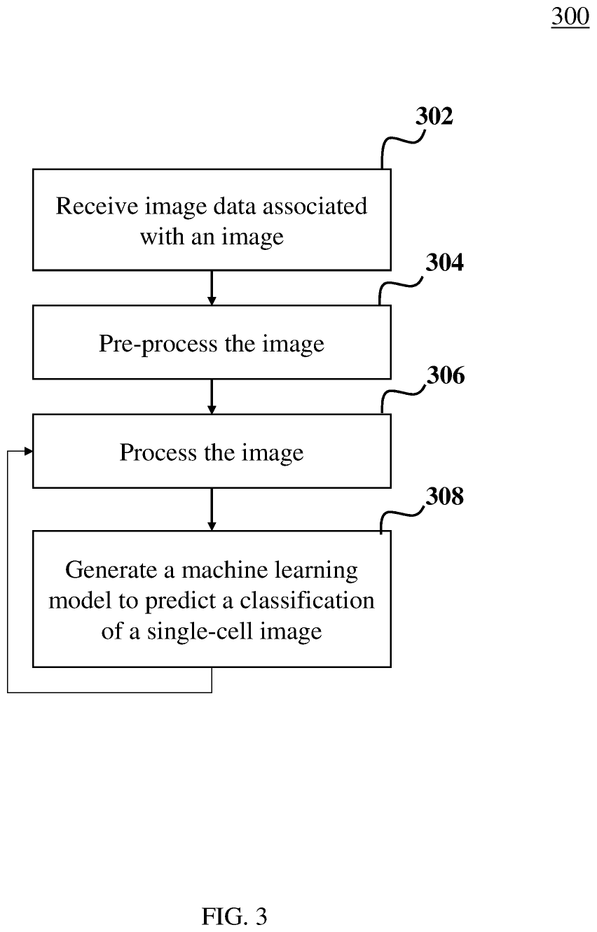

Referring now to FIG. 3, shown is a flow diagram for a non-limiting embodiment or aspect of a process 300 of generating (e.g., training, validating, re-training, and/or the like) a machine learning model to classify diseases based on expansion microscopic images. The steps shown in FIG. 3 are for example purposes only. It will be appreciated that additional, fewer, different, and/or a different order of steps may be used in non-limiting embodiments. In some non-limiting embodiments or aspects, one or more steps of process 300 may be performed (e.g., completely, partially, etc.) by image classification management system 102 (e.g., one or more devices of image classification management system 102). In some non-limiting embodiments or aspects, one or more of the steps of process 300 may be performed (e.g., completely, partially, etc.) by another device or group of devices separate from or including image classification management system 102, such as image procurement system 104 (e.g., one or more devices of image procurement system 104), and/or user device 106 (e.g., one or more devices of user device 106).

As shown at step 302, process 300 includes receiving image data associated with at least one image. For example, image classification management system may receive (e.g., from image procurement system 104) image data associated with at least one image at a first resolution. In some non-limiting embodiments or aspects, the image data may comprise one or more color channels. In some non-limiting embodiments or aspects, the at least one image may have a resolution of 5 nm to 250 nm per pixel. In some non-limiting embodiments or aspects, the at least one image may include an image of a physically expanded sample of a tissue or a cell specimen. The sample may be expanded by permeating the sample with a polymer monomer composition comprising an α, β-unsaturated carbonyl monomer, such as an acrylate, methacrylate, acrylamide, or methacrylamide monomer for producing a water-swellable (co)polymer, and an enal able to polymerize with the acrylate, methacrylate, acrylamide, or methacrylamide monomer; and polymerizing the polymer monomer composition with the enal to form a swellable material containing the cell or tissue sample, resulting in covalent linking of the enal to both the swellable material and a biomaterial in the sample.

As shown at step 304, process 300 includes pre-processing the at least one image. For example, image classification management system 102 may pre-process the at least one image. In some non-limiting embodiments or aspects, pre-processing the at least one image may include removing a background from the at least one image. For example, image classification management system 102 may remove a background from the at least one image. In some non-limiting embodiments or aspects, pre-processing the image may include deconstructing the at least one image into separate layers. For example, image classification management system 102 may deconstruct the at least one image into separate layers based on each of the one or more color channels of the at least one image.

As shown at step 306, process 300 includes processing the at least one image. For example, image classification management system 102 may process the at least one image. In some non-limiting embodiments or aspects, processing the image may include segmenting the at least one image to define a plurality of single-cell images. For example, image classification management system 102 may segment the at least one image to define a plurality of single-cell images. The at least one image may include an image of a plurality of cells. The image of the plurality of cells may include a plurality of nuclei. In some non-limiting embodiments or aspects, segmenting the at least one image to define the plurality of single-cell images may include: identifying a location of each cell of the plurality of cells of the at least one image based on a pixel coordinate of each cell of the plurality of cells of the at least one image; and defining, for each of the plurality of cells, a single-cell image based on the location of a cell of the plurality of cells. The single-cell image may include the cell of the plurality of cells and a microenvironment of the cell of the plurality of cells. In some non-limiting embodiments or aspects, prior to identifying the location of each cell of the plurality of cells of the at least one image, processing the image may include blurring the at least one image. For example, image classification management system 102 may blur the at least one image by decreasing the resolution of the at least one image to facilitate identification of the plurality of nuclei. In some non-limiting embodiments or aspects, processing the image may include applying a filter to at least one single-cell image. For example, image classification management system 102 may apply a filter to at least one single-cell image of the plurality of single-cell images. The filter may decrease a resolution of the at least one single-cell image as compared to the first resolution, to a second resolution. In some non-limiting embodiments or aspects, processing the image may include assigning a label to the at least one single-cell image. For example, image classification management system 102 may assign a label to the at least one single-cell images.

Additionally or alternatively, processing the image may include augmenting the image data associated with the at least one image. For example, image classification management system 102 may augment the image data associated with the at least one image. In some non-limiting embodiments or aspects, augmenting the image data may include manipulating an image perspective of the at least one single-cell image of the plurality of single-cell images to provide a plurality of augmented single-cell images. Manipulating the image perspective may include randomly flipping, randomly rotating, randomly shearing along an axis, and/or randomly translating at least one single-cell image of the plurality of single-cell images, and/or adding random noise to the at least one single-cell image of the plurality of single-cell images.

As shown at step 308, process 300 includes generating (e.g., training, validating, re-training, and/or the like) a machine learning model to predict a classification of a single-cell image. For example, image classification management system 102 may generate a machine learning model to predict a classification of at least one single-cell image. In some non-limiting embodiments or aspects, generating a machine learning model may include training a machine learning model to predict a classification of at least one single-cell image. For example, image classification management system 102 may train a machine learning model to predict a classification of the at least one single-cell image of the plurality of single-cell mages based on inputting the plurality of single-cell images into the machine learning model.

Additionally or alternatively, generating a machine learning model may include determining whether an accuracy value of the machine learning model is above a threshold value. For example, image classification management system 102, may determine whether an accuracy value of the machine learning model is above a threshold value. The threshold value may be a predetermine value and/or a value set by a user. Based on determining that the accuracy value of the machine learning model is above a threshold value, generating the machine learning model may include determining whether a classification predicted for the at least one single-cell image matches a classification of the at least one single-cell image. For example, image classification management system 102 may determine whether a classification predicted for the at least one single-cell image matches the label assigned (e.g., by the image classification management system 102) to the at least one single-cell image. Based on determining that a classification predicted for the at least one single-cell image does not match a classification of the at least one single-cell image, determining that the label predicted for the at least one single-cell image does match a classification of the at least one single-cell image, process 300 may include automatically correcting a classification of the at least one single-cell image to match the classification predicted for the at least one single-cell image. For example, image classification management system 102 may correct a classification of the at least one single-cell images to match the classification predicted for the at least one single-cell image.

Additionally or alternatively, process 300 may include repeating the steps of: applying the filter to the at least one single-cell image of the plurality of single-cell images, wherein the filter increases the resolution of the at least one single-cell image of the plurality of single-cell images as compared to the second resolution and below the first resolution; assigning the label to the at least one single-cell image of the plurality of single-cell images; and training the machine learning model to predict a classification of the at least one single-cell image of the plurality of single-cell images. In some non-limiting embodiments or aspects, process 300 may include repeating, one or more additional times, the steps of: applying the filter to the at least one single-cell image of the plurality of single-cell images, wherein the filter increases the resolution of the at least one single-cell image of the plurality of single-cell images as compared to the second resolution and below the first resolution; assigning the label to the at least one single-cell image of the plurality of single-cell images; and training the machine learning model to predict a classification of the at least one single-cell image of the plurality of single-cell images, wherein with each subsequent repeat of the steps, the resolution of the filter increases from a previous repetition towards or to the first resolution. In some non-limiting embodiments or aspects, the steps may be repeated until the resolution of the at least one single-cell image of the plurality of single-cell images is at the first resolution of the plurality of single-cell images. In some non-limiting embodiments or aspects, the steps may be repeated until the resolution of the at least one single-cell images of the plurality of single-cell images is a highest resolution.

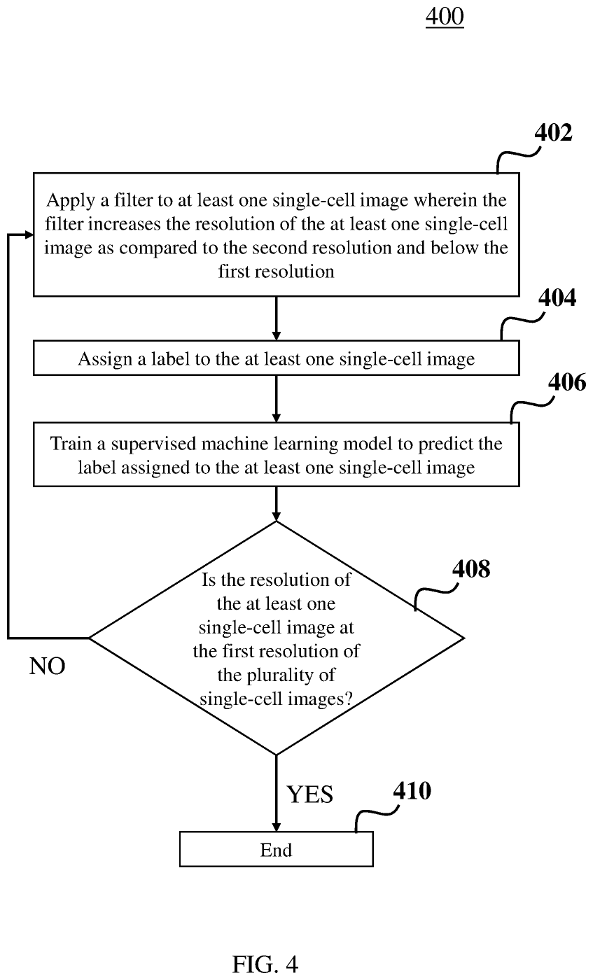

Referring now to FIG. 4, shown is an exemplary flowchart of an implementation of a non-limiting embodiment or aspect of the process shown in FIG. 3. The steps shown in FIG. 4 are for example purposes only. It will be appreciated that additional, fewer, different, and/or a different order of steps may be used in non-limiting embodiments. In some non-limiting embodiments or aspects, one or more steps of process 400 may be performed (e.g., completely, partially, etc.) by image classification management system 102 (e.g., one or more devices of image classification management system 102). In some non-limiting embodiments or aspects, one or more of the steps of process 400 may be performed (e.g., completely, partially, etc.) by another device or group of devices separate from or including image classification management system 102, such as image procurement system 104 (e.g., one or more devices of image procurement system 104), and/or user device 106 (e.g., one or more devices of user device 106).

As shown at step 402, process 400 includes applying a filter to at least one single-cell image. For example, image classification management system 102 may apply a filter to the at least one single-cell image. The filter may increase the resolution of the at least one single-cell image as compared to the second resolution and below the first resolution. The filter may be any type of filter used for image signal processing (e.g., a Gaussian filter).

As shown at step 404, process 400 includes assigning a label to the at least one single-cell image. For example, image classification management system 102 may assign a label to the at least one single-cell image.

As shown at step 406, process 400 includes generating (e.g., training and validating) a supervised machine learning model to predict a classification of the at least one single-cell image. For example, image classification management system 102 may input (e.g., into ML model system 106) a plurality of single-cell images to train the machine learning model to predict a classification of at least one single-cell image.

As shown at step 408, process 400 includes determining whether the resolution of the at least one single-cell image is at the first resolution of the plurality of single-cell images. In some non-limiting embodiments or aspects, in response to determining that the resolution of the at least one single-cell image is not at the first resolution of the plurality of single-cell images, process 400 proceeds to step 402. In some non-limiting embodiments or aspects, in response to determining that the resolution of the at least one single-cell image is at the first resolution of the plurality of single-cell images, process 400 proceeds to step 410 and process 400 ends.

### Example

Acquisition of diffraction unlimited fluorescent images of human specimens: Ultrahigh resolution images can be acquired by imaging labelled regions of interest from human tissue sections or cytology slides using any suitable super-resolution imaging techniques, e.g., as described in. The resolution of images should be beyond optical diffraction limit under that given microscopic setup or better than 100 nm per pixel.

Image segmentation: Individual cells and their surroundings that have implication in the microenvironment are first segmented using blob analysis. Images with large field of view are loaded through a custom reader. A Gaussian filter with a sigma value of ranging from 10-100 is applied to the images to remove high resolution content that might confuse segmentation. The coordinates of the segmented cells are saved in a separate file in case the user wishes to apply different sigma value for the Gaussian filter to the original images. The image processing of this pipeline entails the segmentation of the cells along with the microenvironment around it, allowing the network to take into consideration more information that may be relevant to the character of the cell. As part of the preprocessing, the pipeline also allows input of fluorescent images with as many channels as possible so long as the memory of the workstation permits for the network to use to make a better classification.

Modified ResNet: ResNet is a residual network with proven performance and strong capability of pattern recognition through classification of the ImageNet dataset. The residual network used in the pipeline is a 50-layer ResNet where the 2-layer blocks in the 34-layer residual network are replaced by 3-layer bottleneck blocks. The input size of the network has been changed from a fixed 224×224×3 pixel input to a variable input that automatically detects the dimensions of the image data to be fed into the network. The input data consists of images of a single expanded cell with pre-defined number of layers and the network outputs a decision value based on the probability that the cell in the image is one of the five classes: normal, cancerous, other, trash or undetermined.

Ground Truth: The residual network trained in the pipeline is in the form of supervised learning, which requires a ground truth dataset of the training examples. The images to be used as training examples were segmented cell images from labeled regions of interest. To keep a balanced dataset to prevent the introduction of any bias to the training process, the number of training images in each class is kept the same to each other.

Data Augmentation: Data augmentation was performed on the dataset to increase the size of training data and reduce the chance of overfitting. The augmentation operation includes random flipping, random rotations, random shearing along an axis, random translation either horizontally or vertically.

Training through Deep Learning: The structure of the 50-layer residual network was adopted and used for image classification by training a network from scratch. The implementation of the residual network was done utilizing the deep learning toolbox available as an add-on in MATLAB. The residual network was trained on Windows 10 using four NVIDIA RTX 2080 TI graphics cards. For example, the network is trained for 100 epochs at a mini batch size of 32, with an initial learning rate of 0.0001. The learning rate is adjusted by a factor of 0.7 every 20 epochs. The stochastic gradient descent optimizer was used with a momentum of 0.85, and L2 regularization was performed with a factor of 0.005. For nanoscale images, Gaussian filters with kernel size 2 are applied to the raw images in the first training. Multiple fine-tuning processes are followed. In each fine-tuning cycle, the kernel size of Gaussian filters is reduced sequentially, which forces the model to learn low resolution information first before focusing on the nanoscale details and thus avoids having the model stuck in the local optimal and dramatically increase the classification accuracy. If trained model has high accuracy (>90%) in validation dataset, optionally, iterative rounds of self-correction can be utilized to correct mislabeled images and further improve the model accuracy in the fine-tuning steps. For example, between each fine-tuning process, the trained network would be used to identify a small subset of images classified with 99% probability as different category than original label. This subset of images is defined as incorrect or inaccurate labels and their labels will be replaced with the label of higher probability.

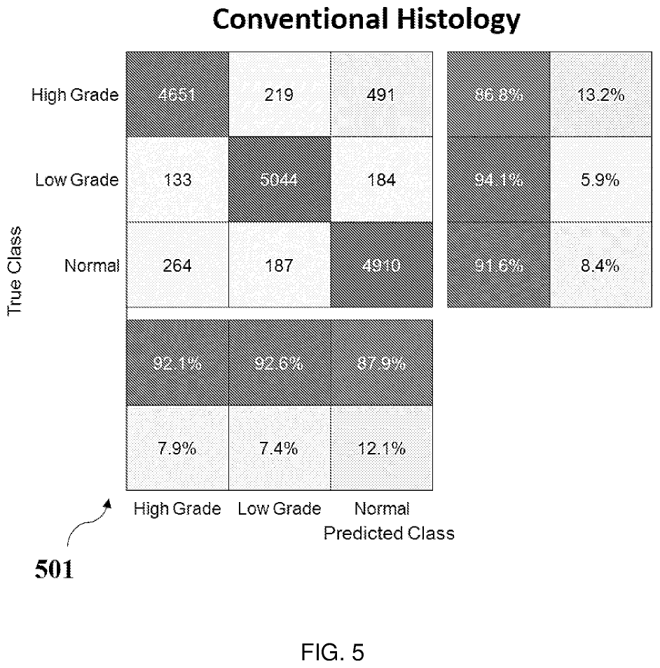

Example results on classification of histology and cytology samples are shown in FIGS. 5-8. Referring now to FIGS. 5-8, shown are exemplary images of confusion matrices illustrating the training results of a machine learning model generated (e.g., trained and validated) using the process shown in FIG. 3.

FIG. 5 is an exemplary image 501 of a confusion matrix of the training results of a dataset (e.g., H&E dataset). As shown in FIG. 5, the training accuracy (e.g., accuracy value) for the H&E dataset is 91.2%.

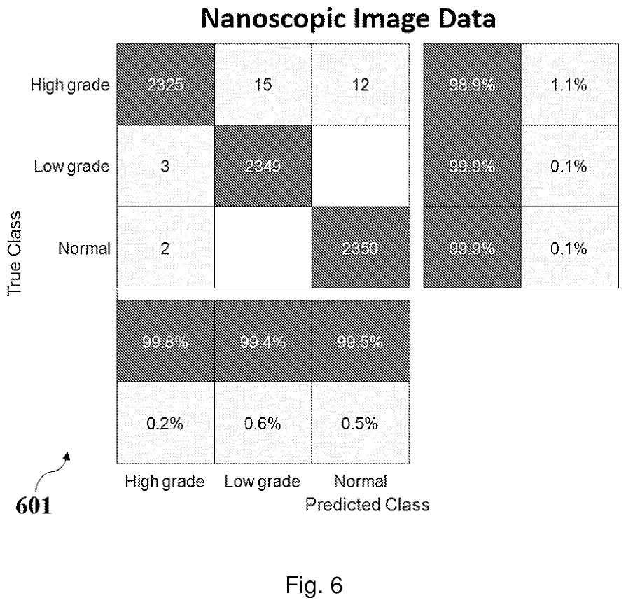

FIG. 6 is an exemplary image 601 of a confusion matrix of the training results of a data set (e.g., expanded tissue image dataset). The expanded tissue data set may include images expanded using methods, systems, and/or products as described herein. As shown in FIG. 6, the training accuracy (e.g. accuracy value) of the expanded tissue dataset is 99.3%

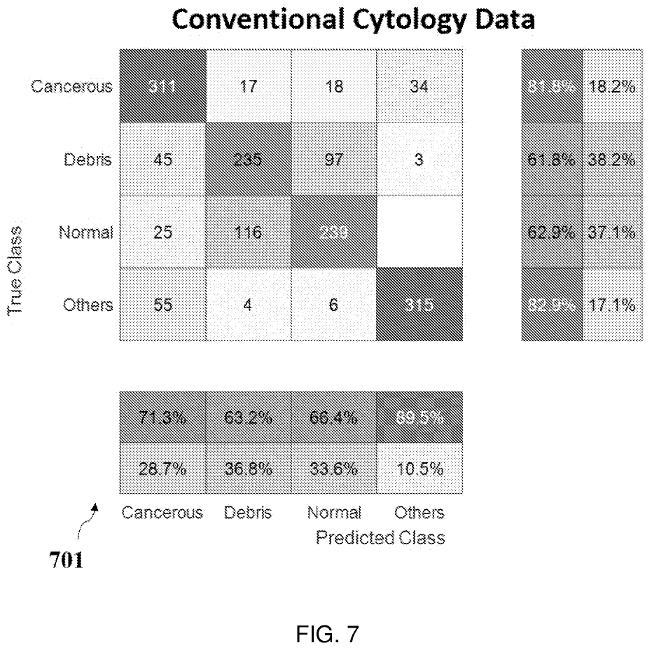

FIG. 7 is an exemplary image 701 of a confusion matrix of the training results of a dataset (e.g., cytology image dataset). As seen in FIG. 7, the cytology image dataset has a training accuracy (e.g., accuracy value) of 72%.

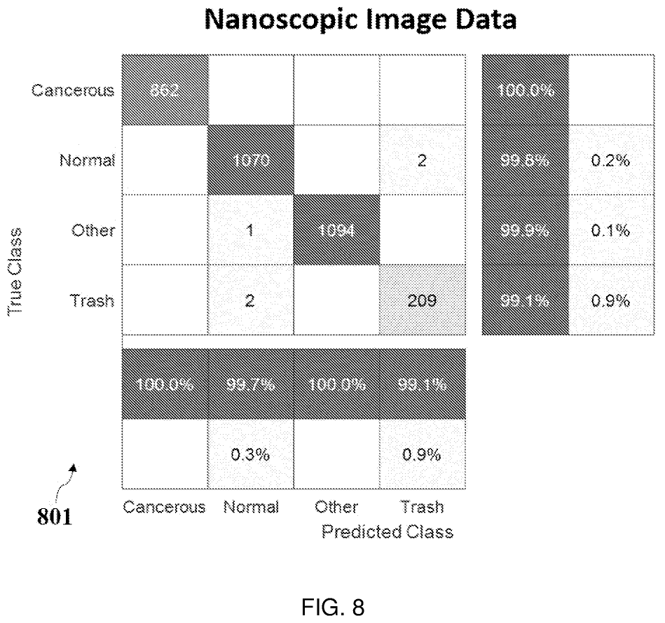

FIG. 8 is an exemplary image 801 of a confusion matrix of the training results of a data set (e.g., expanded cytology image dataset). The expanded cytology dataset may include images expanded using methods, systems, and/or products as described herein. As shown in FIG. 8, the training accuracy (e.g. accuracy value) of the expanded cytology dataset is 99.8%.

As the deep learning toolbox on MATLAB provides other neural networks such as GoogLeNet, VGG, MobileNet, etc., the user can customize the network to use to check if a neural network other than ResNet50 yields better results.

Classification and Output: With the trained network, tissue images from patients can be segmented and classified. The classified images can be grouped and assigned to the subfolder of the classified category. This way helps user to organize the results of image classification and collect more example images for fine-tuning. The results may be organized may be exported to an Excel file with their relative patient codes for labeling.

The segmented images from the same region of interest can also be used to predict the disease type or treatment outcome of the patient. For calling the disease type or treatment outcome from a given region of interest, the framework assumes all the cells from the given region of interest are derived from the same origin/clone. Therefore, the framework only calls the disease type of treatment outcome that gets the most hit from the classification of individual segmented images.

The present invention has been described with reference to certain exemplary embodiments, dispersible compositions and uses thereof. However, it will be recognized by those of ordinary skill in the art that various substitutions, modifications or combinations of any of the exemplary embodiments may be made without departing from the spirit and scope of the invention. Thus, the invention is not limited by the description of the exemplary embodiments, but rather by the appended claims as originally filed.

1. A computer implemented method comprising:
receiving, with at least one processor, image data associated with at least one image at a first resolution;
removing, with the at least one processor, a background from the at least one image;
segmenting, with the at least one processor, the at least one image to define a plurality of single-cell images;
applying, with the at least one processor, a filter to at least one single-cell image of the plurality of single-cell images, wherein the filter decreases a resolution of the at least one single-cell image as compared to the first resolution, to a second resolution;
assigning, with the at least one processor, a label to the at least one single-cell image of the plurality of single-cell images; and
training, with the at least one processor, a machine learning model to predict a classification of the at least one single-cell image of the plurality of single-cell images based on inputting the plurality of single-cell images into the machine learning model.

2. The computer implemented method of claim 1, wherein the at least one image comprises an image of a plurality of cells, and wherein segmenting the at least one image to define the plurality of single-cell images comprises:
identifying a location of each cell of the plurality of cells of the at least one image based on a pixel coordinate of each cell of the plurality of cells of the at least one image; and
defining for each of the plurality of cells, a single-cell image based on the location of a cell of the plurality of cells, wherein the single-cell image comprises the cell of the plurality of cells and a microenvironment of the cell of the plurality of cells.

3. The computer implemented method of claim 2, wherein the image of the plurality of cells comprises a plurality of nuclei, and wherein prior to identifying the location of each cell of the plurality of cells of the at least one image, the method further comprises:
blurring the at least one image by decreasing the resolution of the at least one image to facilitate identification of the plurality of nuclei.

4. The computer implemented method of claim 1, further comprising:
repeating, the steps of:
applying the filter to the at least one single-cell image of the plurality of single-cell images, wherein the filter increases the resolution of the at least one single-cell image of the plurality of single-cell images as compared to the second resolution and below the first resolution;
assigning the label to the at least one single-cell image of the plurality of single-cell images; and
training the machine learning model to predict a classification of the at least one single-cell image of the plurality of single-cell images.

5. The computer implemented method of claim 4, further comprising repeating one or more additional times, the steps of:
applying the filter to the at least one single-cell image of the plurality of single-cell images;
assigning the label to the at least one single-cell image of the plurality of single-cell images; and
training the machine learning model to predict a classification of the at least one single-cell image of the plurality of single-cell images,
wherein with each subsequent repeat of the steps, the resolution of the filter increases from a previous repetition towards or to the first resolution.

6. The computer implemented method of claim 5, wherein the steps are repeated until the resolution of the at least one single-cell image of the plurality of single-cell images is at the first resolution of the plurality of single-cell images.

7. The computer implemented method of claim 6, wherein the steps are repeated until the resolution of the at least one single-cell image of the plurality of single-cell images is a highest resolution.

8. The computer implemented method of claim 1, wherein the filter is a Gaussian filter.

9. The computer implemented method of claim 1, wherein the machine learning model outputs a classification value for the at least one single-cell image of the plurality of single-cell images, and wherein training the machine learning model to predict a classification of the at least one single-cell image of the plurality of single-cell images further comprises:
determining whether an accuracy value of the machine learning model is above a threshold value;
based on determining that the accuracy value of the machine learning model is above the threshold value, determining whether the classification predicted for the at least one single-cell image matches a classification of the at least one single-cell image; and
based on determining that the classification predicted for the at least one single-cell image does not match a classification of the at least one single-cell image, automatically correcting the classification of the at least one single-cell image to match the classification predicted for the at least one single-cell image.

10. The computer implemented method of claim 1, further comprising:
manipulating an image perspective of the at least one single-cell image of the plurality of single-cell images to provide a plurality of augmented single-cell images, wherein manipulating the image perspective comprises randomly flipping, randomly rotating, randomly shearing along an axis, and/or randomly translating at least one single-cell image of the plurality of single-cell images, and/or adding random noise to the at least one single-cell image of the plurality of single-cell images.

11. The computer implemented method of claim 1, wherein the at least one image has a resolution of 5 nm to 250 nm per pixel.

12. The computer implemented method of claim 1, wherein the machine learning model comprises a convolutional neural network (CNN).

13. The computer implemented method of claim 1, wherein the at least one image comprises an image of a physically expanded sample of a tissue or a cell specimen.

14. The computer implemented method of claim 13, wherein the sample is expanded by permeating the sample with a polymer monomer composition comprising an α, β-unsaturated carbonyl monomer, such as an acrylate, methacrylate, acrylamide, or methacrylamide monomer for producing a water-swellable (co)polymer, and an enal able to polymerize with the acrylate, methacrylate, acrylamide, or methacrylamide monomer; and polymerizing the polymer monomer composition with the enal to form a swellable material containing the cell or tissue sample, resulting in covalent linking of the enal to both the swellable material and a biomaterial in the sample.

15. A system comprising at least one processor programmed or configured to:
receive image data associated with at least one image at a first resolution;
remove a background from the at least one image;
segment the at least one image to define a plurality of single-cell images;
apply a filter to at least one single-cell image of the plurality of single-cell images, wherein the filter decreases a resolution of the at least one single-cell image;
assign a label to the at least one single-cell image of the plurality of single-cell images; and
train a machine learning model to predict a classification of the at least one single-cell image of the plurality of single-cell images based on inputting the plurality of single-cell images into the machine learning model.

16. The system of claim 15, wherein the at least one image comprises an image of a plurality of cells, and wherein when segmenting the at least one image to define the plurality of single-cell images, the at least one processor is further programmed or configured to:
identify a location of each cell of the plurality of cells of the at least one image based on a pixel coordinate of each cell of the plurality of cells of the at least one image; and
define for each of the plurality of cells, a single-cell image based on the location of a cell of the plurality of cells, wherein the single-cell image comprises the cell of the plurality of cells and a microenvironment of the cell of the plurality of cells.

17. The system of claim 16, wherein the image of the plurality of cells comprises a plurality of nuclei, and wherein prior to identifying the location of each cell of the plurality of cells of the at least one image, the at least one processor is further programmed or configured to:
blur the at least one image by decreasing the resolution of the at least one image to facilitate identification of the plurality of nuclei.

18-26. (canceled)

27. The system of claim 15, wherein the at least one image comprises an image of a physically expanded sample of a tissue or a cell specimen.

28. (canceled)

29. A computer program product comprising at least one non-transitory computer readable medium including one or more instructions that, when executed by the at least one processor, cause the at least one processor to:
receive image data associated with at least one image at a first resolution;
remove a background from the at least one image;
segment the at least one image to define a plurality of single-cell images;
apply a filter to at least one single-cell image of the plurality of single-cell images, wherein the filter decreases a resolution of the at least one single-cell image;
assign a label to the at least one single-cell image of the plurality of single-cell images; and
train a machine learning model to predict a classification of the at least one single-cell image of the plurality of single-cell images based on inputting the plurality of single-cell images into the machine learning model.

30-42. (canceled)

43. The computer implemented method of claim 1, comprising:
receiving, with at least one processor, image data associated with at least one image at a first resolution;
removing, with the at least one processor, a background from the at least one image;
segmenting, with the at least one processor, the at least one image to define a plurality of single-cell images;
inputting, with the at least one processor, at least one single-cell image of the plurality of single-cell images into a trained machine learning model, wherein the trained machine learning model outputs a classification value corresponding to a category of a plurality of categories;
sorting, with the at least one processor, the at least one single-cell image of the plurality of single-cell images into one category of the plurality of categories based on the value output by the trained machine learning model;
generating, with the at least one processor, a communication comprising a predicted treatment outcome based on which category of the plurality of categories the at least one single-cell image of the plurality of single-cell images is sorted into; and
displaying, with the at least one processor, data associated with the communication via a graphical user interface (GUI) on a user device,
wherein the trained machine learning model is trained by:
receiving, with at least one processor, image data associated with at least one image at a first resolution;
removing, with the at least one processor, a background from the at least one image;
segmenting, with the at least one processor, the at least one image to define a plurality of single-cell images;
applying, with the at least one processor, a filter to at least one single-cell image of the plurality of single-cell images, wherein the filter decreases a resolution of the at least one single-cell image as compared to the first resolution, to a second resolution;
assigning, with the at least one processor, a label to the at least one single-cell image of the plurality of single-cell images; and
training, with the at least one processor, a machine learning model to predict a classification of the at least one single-cell image of the plurality of single-cell images based on inputting the plurality of single-cell images into the machine learning model.

44-76. (canceled)
